SEQUENCE LISTING (5 sequences)

SEQ ID NO: 1 - DNA, 1260 bp, Hyphomicrobium EG
  CDS (1)..(1260)
atggaggagcaatactttccattgcaggtctatcgccaaggtccttacgctgccggcggcacaggctttttcggtggcatgatcgactacatgaacctgatcaacatgcgcgacggcggcgtcaacggcgtcaagctgacgtggagcgagtgcgagacgggttacgaagtcgaaaagggcgtcgagtgctaccagcgaatgaaggatcacaatcctcctccgggcgtctggacccctctcagcgtcggcatctcttacgctatgactgataagatcacagctgaccaggttcctctgacgacgatcaaccacggtcgcaccgagtcctccgacggacgggtgttcaaatatctcttcccgcttcttacaaactccggcagcgaaacgtccgctatcgtccagtacatcggctcgaaactcggcggtatggagaatctcaaagggaaaaagatcgtcatccttttccacggctctccctatggcaaagaaacgaccccgctattgaaacagctgggtgagaagtacggtttcgaggtgaaggacatcgaagtcccgcacccgggcgccgaacagggatcgcagtggcttgaaatcaagcgctaccgtccggacttcgttattctccgcggttggggaattatgaacccggtggcactcaagacggcacgccgtaccggatatccgatcgataaaatcgtcggcaacatctggtcgaattcggaagaagacgtgaaacccgctggtgatgcaggtatcggctataccgccgttacgacgcagacgtcaggtacgtcctcgccgatcctccaggacattatcaagaccgtctatggtgcaggcaaaggcgaccttgccgataaatcgcgcatcggcagcgtctatcacaaccttggcattgtgaacggcatgctgaccatcgaagcaatgcgcattgcacagaagaagttcggcaacaaaatgctgaccggcgatcagttccgctggggcttcgagcatctgaaactcgatgaagcagctgtcgaaaagatcggtgcaaaaggcttgttccattcgatcaacgtgacgtgcgaagatcacgaaggtaacggcctgctcatcttccagaagtgggatggctcgaagtacgtgccggtcaccgactggatcaagccggactgggcgatgcttcgcccgatcatcgaaacggcctcggccgcttatgcgaaggaaaagggtattactcctcgcaattgcgccgaagaagaaaagcaagcacagaac

SEQ ID NO: 2 - protein, 420 aa, Hyphomicrobium EG
MEEQYFPLQVYRQGPYAAGGTGFFGGMIDYMNLINMRDGGVNGVKLTWSECETGYEVEKGVECYQRMKDHNPPPGVWTPLSVGISYAMTDKITADQVPLTTINHGRTESSDGRVFKYLFPLLTNSGSETSAIVQYIGSKLGGMENLKGKKIVILFHGSPYGKETTPLLKQLGEKYGFEVKDIEVPHPGAEQGSQWLEIKRYRPDFVILRGWGIMNPVALKTARRTGYPIDKIVGNIWSNSEEDVKPAGDAGIGYTAVTTQTSGTSSPILQDIIKTVYGAGKGDLADKSRIGSVYHNLGIVNGMLTIEAMRIAQKKFGNKMLTGDQFRWGFEHLKLDEAAVEKIGAKGLFHSINVTCEDHEGNGLLIFQKWDGSKYVPVTDWIKPDWAMLRPIIETASAAYAKEKGITPRNCAEEEKQAQN

SEQ ID NO: 3 - DNA, 1293 bp, Hyphomicrobium EG
cgagcatatggaagaacagtatttcccattacaagtatatcgtcaaggcccttatgcagcaggtggtaccggcttcttcggtggtatgatcgactatatgaatctgatcaatatgcgcgatggcggcgtcaatggcgtcaagctgacctggagcgaatgtgagactggttacgaggttgaaaagggcgtggagtgctaccagcgcatgaaggaccataatccgccaccgggcgtctggaccccgctgagcgtgggtattagctatgcgatgaccgacaagattacggcagaccaggttccgctgaccaccatcaatcatggccgtacggagagcagcgatggtcgtgttttcaaatacctgtttccgttgctgaccaacagcggtagcgaaaccagcgcaattgttcaatacatcggtagcaagctgggtggtatggagaatctgaaaggcaagaaaatcgttatcctgttccacggctccccgtacggtaaagaaaccacgccgctgctgaaacaactgggcgagaagtacggttttgaggttaaagacatcgaagtcccgcacccaggtgccgagcagggcagccaatggctggagatcaaacgttatcgtccggactttgtgattttgcgtggttggggtattatgaatccggttgcgctgaaaacggcacgccgcaccggttacccgatcgataagattgtgggtaacatttggagcaactctgaagaggatgttaagccggcaggcgatgctggcatcggttacacggcggtgaccactcaaacgtctggtacttccagcccgattctgcaggatatcattaagaccgtgtacggtgccggcaagggcgacttggcggacaaaagccgtattggttcggtgtatcacaacctgggtatcgtcaacggtatgctgaccattgaggcgatgcgtattgcgcagaaaaagtttggcaataagatgctgacgggtgaccagttccgctggggcttcgagcacctgaagctggacgaggctgccgtcgaaaagatcggtgcgaagggcctgtttcacagcatcaacgtgacgtgcgaggaccatgaaggtaacggtttgctgatttttcaaaaatgggatggctccaaatatgttccggtcaccgattggattaaaccggattgggcgatgttgcgtccgattatcgaaacggccagcgctgcgtacgcgaaagagaaaggtatcaccccgcgtaactgtgccgaagaagagaaacaggcgcagaactaaaggcgcgcccgggaagcttctcg

SEQ ID NO: 4 - protein, 12 aa, Hyphomicrobium EG
MAFSLGVTPSSA

SEQ ID NO: 5 - DNA, 26 bp, Artificial sequence
  stop codon, AscI site and HindIII site
taaaggcgcgcccgggaagcttctcg

(19) United States
(12) Patent Application Publication
(10) Pub. No.: US 2015/0147365 A1
(43) Pub. Date: May 28, 2015

(54) ORAL PREPARATION

(71) Applicant: DSM IP ASSETS B.V., Heerlen (NL)

(72) Inventors: Luppo Edens, Echt (NL); Evert Tjeerd Van Rij, Echt (NL); Marco Jan Lambertus De Groot, Echt (NL)

(21) Appl. No.: 14/402,143

(22) PCT Filed: May 13, 2013
(86) PCT No.: PCT/EP2013/061316

§ 371 Date: Nov. 19, 2014

(30) Foreign Application Priority Data
May 31, 2012 (EP) 12170291.4

(51) Int. Cl. A61K 8/66; C12N 9/02; A61Q 11/00; A61K 8/36; A61K 8/02

(52) U.S. Cl. CPC A61K 8/66; A61K 8/36; A61K 8/0216; A61Q 11/00; C12N 9/0051; C12Y 108/03004; A61K 2800/92
USPC 424/401; 424/50; 424/48; 536/23.2; 435/189; 435/320.1; 435/252.3; 435/325; 435/348; 435/419; 435/254.11; 435/257.2

(57) ABSTRACT

The present invention relates to an oral composition comprising a volatile sulphur compound breath-odour controlling amount of methyl mercaptan oxidase. The oral composition may be used in a method for controlling breath-odour in a host, which method comprises applying the composition to the oral cavity of that host. The invention also provides a polypeptide having methyl mercaptan oxidase activity, wherein said polypeptide is: (a) a polypeptide comprising an amino acid sequence as set out in SEQ ID NO: 2; (b) a polypeptide comprising an amino acid sequence having at least 50% sequence identity with the amino acid sequence of SEQ ID NO: 2; (c) a polypeptide encoded by a polynucleotide comprising the polynucleotide sequence as set out in SEQ ID NO: 1 or SEQ ID NO: 3; or (d) a fragment of a polypeptide as defined in (a), (b) or (c).

### FIELD OF THE INVENTION

The present invention relates to an oral composition. The invention also relates to a method for controlling breath-odour which method comprises: providing the oral composition according to any one of the preceding claims; and applying the composition to the oral cavity. Further provided by the invention is the oral composition for controlling breath-odour and use of the oral composition for use in the manufacture of a medicament for use in controlling breath-odour. The invention also provides a methyl mercaptan oxidase polypeptide, a polynucleotide encoding such a polypeptide, a nucleic acid construct and host cell comprising such a polynucleotide, a method for producing the methyl mercaptan oxidase polypeptide and a composition comprising such a polypeptide.

### BACKGROUND TO THE INVENTION

In most cases (85-90%), bad breath originates in the mouth itself. The intensity of bad breath differs during the day, due to eating certain foods (such as garlic, onions, meat, fish and cheese), obesity, smoking and alcohol consumption. Since the mouth is exposed to less oxygen and is inactive during the night, the odor is usually worse upon awakening (“morning breath”). Bad breath may be transient, often disappearing following eating, brushing one's teeth, flossing, or rinsing with specialized mouthwash. Bad breath may also be persistent (chronic bad breath), which is a more serious condition, affecting some 25% of the population in varying degrees.

Most reports now agree that the most frequent sources of halitosis (80-90%)) exist within the oral cavity and include bacterial reservoirs such as the dorsum of the tongue, saliva and periodontal pockets, where anaerobic bacteria degrade sulphur-containing amino acids to produce foul-smelling volatile sulphur compounds (VSCs). These VSCs are the predominant elements of oral malodour, although some do believe that other odorous volatiles, such as certain amines and fatty acids, may play a role.

Chronic halitosis is not well understood by most physicians and dentists, so effective treatment is not always easy to find. The following strategies may be suggested:

(i) Gently cleaning the tongue surface twice daily is the most effective way to keep bad breath in control; that can be achieved using a tooth brush, tongue cleaner or tongue brush/scraper to wipe off the bacterial biofilm, debris, and mucus. Brushing a small amount of antibacterial mouth rinse or tongue gel onto the tongue surface will further inhibit bacterial action.

(ii) Eating a healthy breakfast with rough foods helps clean the very back of the tongue.

(iii) Since dry-mouth can increase bacterial buildup and cause or worsen bad breath, chewing sugarless gum can help with the production of saliva, and thereby help to reduce bad breath. Chewing may help particularly when the mouth is dry, or when one cannot perform oral hygiene procedures after meals (especially those meals rich in protein). This aids in provision of saliva, which washes away oral bacteria, has antibacterial properties and promotes mechanical activity which helps cleanse the mouth.

(iv) Gargling right before bedtime with an effective mouthwash is sometimes suggested, although mouthwashes may contain active ingredients that are inactivated by the soap present in most toothpastes. Thus it is recommended to refrain from using mouthwash directly after toothbrushing with paste (also see mouthwashes, below).

(v) Probiotic treatments, specifically Streptococcus salivarius K12 has been shown to suppress malodorous bacteria growth.

However, there has not been a single documented medical case of successfully cured chronic halitosis using any of the currently available mouthwashes. Moreover, mouthwashes often contain antibacterial agents including cetylpyridinium chloride, chlorhexidine which may can cause temporary staining of the teeth, have an unpleasant taste and, importantly, may disturb the grown of beneficial oral bacterial flora. They may also contain alcohol which is a drying agent and may worsen chronic bad breath.

Antibacterial/biocidal activity in oral compositions such as mouth washes and chewing gums should be avoided as they affect the oral ecosystem. For example, chlorhexidine containing mouthwashes diminish the oral nitrate-reducing power by 90% and thereby the levels of blood plasma nitrite levels by 25% resulting in a 2-3.5 mm blood pressure increase (Kapil et al. Free Radical Biology and Medicine 55 (2013) 93-100). This is highly significant since Cook et al. (Archives of Internal Medicine 155(7) (1995) 701-9) estimated that a 2 mm Hg reduction in the population average of diastolic blood pressure for white US subjects aged 35-64 years old would result in a 17% decrease in the prevalence of hypertension, a 15% reduction in the risk of stroke, and a 6% reduction in the risk of coronary heart disease.

Although bad breath primarily represents a source of embarrassment or annoyance, the VSCs most responsible for halitosis are also potentially damaging to the tissues in the mouth, and can lead to periodontitis (inflammation of the gums and ligaments supporting the teeth). In particular, VSCs have been found to damage the collagen and proteoglycan components in connective tissue by cleaving disulfide bonds. This de-aggregation of the extracellular matrix allows microbes to permeate the oral mucosa. As bacteria further accumulates in pockets that form next to the teeth, periodontal disease progresses, as well as halitosis. If the periodontal disease advances significantly, overall systemic health may be jeopardized; for example, periodontal bacterial by-products can enter the blood stream and may result in heart disease, stroke and under-weight babies at birth.

Clearly then halitosis has both cosmetic and health implications.

There is thus a need to for improved oral compositions which may be used in the control of bad breath and to avoid associated health problems. Ideally, these compositions should be taste neutral, avoid teeth staining and be non-antibacterial.

### SUMMARY OF THE INVENTION

The current invention relates to a composition for controlling breath-odour, in particular to the control of volatile sulphur compounds (VSCs). A strong correlation has been found between the degree of intra-oral halitosis as measured by organoleptic scoring of mouth breath and the concentration of the VSCs hydrogen sulphide (H₂S) and methyl mercaptan (CH₃SH) in mouth breath.

According to the invention, there is thus provided an oral composition comprising a volatile sulphur compound breath-odour controlling amount of methyl mercaptan oxidase (MM-oxidase). However, this enzyme has not been cloned and no sequence information is available; rather, the enzyme has only ever been isolated from cell cultures. Culture of the species of microorganisms known to express MMO-oxidase is not trivial and requires the use of non-standard media and foul smelling or toxic substrates. Accordingly, there is currently no convenient, cost-effective source of MMO-oxidase which could be used in a preparation of the invention. In view of this, herein is described a strategy for identification of the gene sequence of MMO-oxidases and for their expression in readily cultivatable microorganisms.

Using of this strategy has enabled identification of a novel MM-oxidase enzyme. Although the genomes of several sulphur bacteria have been sequenced, none of them incorporates the MM-oxidase gene sequence as identified herein (having the amino acid set out as SEQ ID NO: 2, also referred to as HDEA00816 in the Examples). Access to the sequence of this enzyme allows it to be produced using conventional enzyme expression techniques, providing a convenient source of MM-oxidase.

Thus, the invention provides a MM-oxidase containing composition which may readily be produced in a cost-effective fashion. The MM-oxidase used in a composition of the invention may be any MM-oxidase, for example the MM-oxidase described herein.

Accordingly, the invention provides an oral composition comprising a volatile sulphur compound breath-odour controlling amount of methyl mercaptan oxidase. Enzyme compositions are known to be non-antibacterial/non-biocidal (with a few well-known exceptions such as chloroperoxidase and lactoperoxidase). Accordingly, MM-oxidase compositions of the invention are advantageous since they do not disturb the oral bacterial flora. Furthermore, such compositions are tasteless, unlike, for example, allyl isothiocyanate which is proposed for use in oral compositions and which has a strong wasabi taste.

The invention further provides a polypeptide having methyl mercaptan oxidase activity, wherein said polypeptide is:
(a) a polypeptide comprising an amino acid sequence as set out in SEQ ID NO: 2;
(b) a polypeptide comprising an amino acid sequence having at least 50% sequence identity with the amino acid sequence of SEQ ID NO: 2;
(c) a polypeptide encoded by a polynucleotide comprising the polynucleotide sequence as set out in SEQ ID NO: 1 or SEQ ID NO: 3; or
(d) a fragment of a polypeptide as defined in (a), (b), (c) or (d).

Such a polypeptide may be used in an oral composition of the invention.

The invention also provides:
a polynucleotide encoding the polypeptide according to the invention;
a nucleic acid construct comprising a polynucleotide of the invention;
a host cell comprising the polynucleotide or nucleic acid construct of the invention;
a method of producing a polypeptide of the invention, comprising:
(a) cultivating the host cell of the invention under conditions conducive to the production of the polypeptide by the host cell; and, optionally,
(b) recovering the polypeptide;
a composition, such as an oral composition, comprising a polypeptide of the invention and at least one additional ingredient;
a method for controlling breath-odour in a host which method comprises:
   providing a composition of the invention, for example one which comprises a polypeptide according to the invention; and
   applying the composition to the oral cavity of the host;
an oral composition of the invention for controlling breath-odour; and
use of an oral composition of the invention for use in the manufacture of a medicament for use in controlling breath-odour;

### DESCRIPTION OF THE FIGURES

FIG. 1 sets out SDS-PAGE of DMSO-grown Hyphomicrobium EG cells lysate purified by DEAE and hydroxylapatite chromatography. Lane 1: molecular weight marker; lane 2: cell extract after DEAE chromatography; lanes 3 and 4: flow-through of hydroxyapatite column; lanes 5, 6 and 7: peak fractions of first protein peak eluted from hydroxyapatite column; lanes 8, 9 and 10: peak fractions of second protein peak eluted from hydroxyapatite column.

FIG. 2 sets out ethyl mercaptan oxidase activity present in the lysates of various E. coli clones MMO1 (‘101’), MMO2 (‘102’, three different transformants), MMO3 (‘103) and MMO4 (‘104). ‘Ref’ refers to E. coli lysates from transformants without a Hyphomicrobium gene insert. Enzyme activity units: 1 unit=1 micromole of ethyl mercaptane converted per minute at 30° C.

FIG. 3 sets out degradation over time of methyl mercaptan by different hydroxyapatite enzyme fractions mixed with Zendium toothpaste as assayed by headspace analysis.

### DESCRIPTION OF SEQUENCE LISTING

SEQ ID NO: 1 sets out the nucleotide sequence of the open reading frame named MMO2

SEQ ID NO: 2 sets out the amino acid sequence of HDEA00816 protein (MMO2), without the putative signal peptide MAFSLGVTPSSA.

SEQ ID NO: 3 sets out the nucleotide sequence of the E. coli optimized MMO2 gene (named MMO2E) with NdeI site introduced at the 5′-end and a stop codon and AscI and HindIII sites introduced at the 3′end.

### DETAILED DESCRIPTION OF THE INVENTION

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element.

The invention relates to an oral composition to control breath-odour. That is to say, the oral composition of the invention is intended to reduce the amounts of one or more volatile sulphur compounds (VSCs) that may contribute to breath-odour. Thus, in the context of this invention, a volatile sulphur compound breath-odour controlling amount of methyl mercaptan oxidase is an amount which reduces the presence of one or more VSCs, so that breath-odour is reduced (for example as determined chemically or oganoleptically).

Herein, the terms breath-odour, halitosis, malodour and the like are all intended to indicate the presence of odourous VSCs.

The term “oral malodour” has been used in the literature to refer to bad breath originating from the mouth. However, Tangerman and Winkel (J Clin. Perodontol. 34, 748-755, 2007) draw a distinction between use the term “intra-oral halitosis” instead of oral malodour, so as to differentiate it from extra-oral halitosis. The distinction between intra- and extra-oral halitosis may be carried out by comparing mouth breath with nose breath.

Tangerman and Winkel argue that in oral halitosis, the presence of the VSC methyl mercaptan, MM (CH₃SH), in mouth breath is the predominant causative factor of oral malodour, more so than H₂S.

The composition of the invention comprises a methyl mercaptan oxidase (MM-oxidase). MM-oxidase is any enzyme that has the activity of being able to oxidize methyl mercaptan. That is to say, MM-oxidase (EC 1.8.3.4) is any enzyme that catalyzes the chemical reaction:

methanethiol+O₂+H₂O [symbol] formaldehyde+hydrogen sulfide+H₂O₂

The 3 substrates of this enzyme are methanethiol (methyl mercaptan), O₂, and H₂O, whereas its 3 products are formaldehyde, hydrogen sulphide, and H₂O₂.

The enzyme belongs to the family of oxidoreductases, specifically those acting on a sulfur group of donors with oxygen as acceptor. The systematic name of this enzyme class is methanethiol:oxygen oxidoreductase. An enzyme suitable for use in the invention may be referred to as methanethiol oxidase, methylmercaptan oxidase, methyl mercaptan oxidase, (MM)-oxidase or MT-oxidase.

An MM-oxidase suitable for use in the invention may be any MM-oxidase from any source. An oral composition of the invention may comprise a methyl mercaptan oxidase derived or derivable from a Hyphomicrobium species, in particular Hyphomicrobium EG, from a Rhodococcus species such as Rhodococcus rhodochrous or a Thiobacillus species such as Thiobacillus thioparus.

The term “derived from” herein also includes the terms “originated from,” “obtained from,” “obtainable from,” “isolated from,” and “created from,” and generally indicates that one specified material find its origin in another specified material or has features that can be described with reference to the another specified material. As used herein, a substance (e.g., a nucleic acid molecule or polypeptide) “derived from” a microorganism preferably means that the substance is native to that microorganism.

Classical production of MM-oxidase by any of the aforementioned microbial species is not economically viable. Inter alia because of the toxicity or the bad smell of the relevant substrates as well as because of the very low growth rate of such organisms on the substrate required to maximise production of the desired MM-oxidase activity. Thus, over-expression of the enzyme by recombinant DNA technology will typically be required in order to provide MM-oxidase for the oral composition of the invention.

Herein is described an MM-oxidase which may be expressed by recombinant means. The term “recombinant” when used in reference to a cell, nucleic acid, protein or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. The term “recombinant” is synonymous with “genetically modified”.

Accordingly, the invention provides a polypeptide having MM-oxidase activity. Such a polypeptide may be selected from the group consisting of:
(a) a polypeptide comprising an amino acid sequence as set out in SEQ ID NO: 2;
(b) a polypeptide comprising an amino acid sequence having at least 50% sequence identity with the amino acid sequence of SEQ ID NO: 2;
(c) a polypeptide encoded by a polynucleotide comprising the polynucleotide sequence as set out in SEQ ID NO: 1 or SEQ ID NO: 3; or
(d) a fragment of a polypeptide as defined in (a), (b) or (c).

Such a polypeptide may comprise an amino acid sequence having at least 60% sequence identity, preferably at least about 70%, more preferably at least about 80%, more preferably at least about 85%, even more preferably at least about 90%, even more preferably at least about 93%, even more preferably at least about 95%, even more preferably at least about 96%, preferably at least about 97%, even more preferably at least about 98% and even most preferably at least about 99% sequence identity to SEQ ID NO 2.

That is to say, the invention relates to a polypeptide comprising an amino acid sequence of SEQ ID NO: 2 and to variants thereof (having the sequence identity with SEQ ID NO: 2 as set out above). As used herein, the terms “variant, “derivative”, “mutant” or “homologue” can be used interchangeably. They can refer to either polypeptides (or to the nucleic acids enc. Variants include substitutions, insertions, deletions, truncations, transversions, and/or inversions, at one or more locations relative to a polypeptide comprising the amino acid sequence of SEQ ID NO: 2. Variants can be made for example by site-saturation mutagenesis, scanning mutagenesis, insertional mutagenesis, random mutagenesis, site-directed mutagenesis, and directed-evolution, as well as various other recombination approaches. Variant polypeptides may differ from a reference polypeptide by a small number of amino acid residues and may be defined by their level of primary amino acid sequence homology/identity with a reference polypeptide.

Preferably, variant polypeptides have at least 50%, at least 60%, at least 70% 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a polypeptide comprising the amino acid sequence of SEQ ID NO: 2.

A variant of the invention may be an allelic variant of a polypeptide comprising the amino acid sequence of SEQ ID NO: 2. An “allelic variant of a polypeptide” is herewith defined as a polypeptide encoded by an allelic variant of a gene. The term “allelic variant of a gene” denotes herein any of two or more alternative forms of a gene occupying the same chromosomal locus. An allelic variant of a gene may comprise gene mutations which are silent (resulting in no change in the amino acid sequence of the polypeptide encoded by the gene); it may comprise gene mutations which result in polypeptides having altered amino acid sequences or it may comprise both.

Methods for determining percent identity are known in the art and described herein. Generally, the variants retain the characteristic nature of the reference polypeptide, but have altered properties in some specific aspects. For example, a variant may have a modified pH optimum, a modified substrate binding ability, a modified resistance to enzymatic degradation or other degradation, an increased or decreased activity, a modified temperature or oxidative stability, but retains its characteristic functionality. Variants further include polypeptides with chemical modifications that change the characteristics of a reference polypeptide.

With regard to nucleic acids, the term “variant” may refer to a nucleic acid that encodes a variant polypeptide that has a specified degree of homology/identity with a polynucleotide comprising the nucleic acid sequence set out in SEQ ID NO: 1 or 3. Preferably, a variant polynucleotide has at 50%, at least 60%, at least 70%, at least 75%, at least 80%, at least 85%, at least 90%, at least 91%, at least 92%, at least 93%, at least 94%, at least 95%, at least 96%, at least 97%, at least 98%, or even at least 99% nucleic acid sequence identity with a polynucleotide comprising the nucleic acid sequence set out in SEQ ID NO: 1 or 3. Methods for determining percent identity are known in the art and described herein.

The sequence identities referred to herein may be determined in relation to a mature polypeptide or in relation to a prepropeptide or propeptide. The term “mature polypeptide” is defined herein as a polypeptide in its final form and is obtained after translation of a mRNA into polypeptide and post-translational modifications of said polypeptide. Post-translational modification include N-terminal processing, C-terminal truncation, glycosylation, phosphorylation and removal of leader sequences such as signal peptides, propeptides and/or prepropeptides as defined herein by cleavage. The term “prepropeptide” is defined herein as a signal peptide and propeptide present at the amino terminus of a polypeptide, where the propeptide is linked (or fused) in frame to the amino terminus of a polypeptide and the signal peptide is linked in frame (or fused) to the amino terminus of the propeptide region. The term “signal peptide” is defined herein as a peptide linked (fused) in frame to the amino terminus of a polypeptide and directs the polypeptide into the cell” secretory pathway. A propeptide may be present between the signal peptide and the amino terminus of the polypeptide. The term “propeptide” is an amino acid sequence linked (fused) in frame to the amino terminus of a polypeptide having biological activity, wherein the resultant polypeptide is known as a proenzyme or propolypeptide (or a zymogen in some cases), A propolypeptide is generally biologically inactive and can be converted to a mature active polypeptide by catalytic or autocatalitic cleavage of the propeptide from the propolypeptide.

As used herein, the term “polypeptide” refers to a molecule comprising amino acid residues linked by peptide bonds and containing more than five amino acid residues. The amino acids are identified by either the single-letter or three-letter designations. The term “protein” as used herein is synonymous with the term “polypeptide” and may also refer to two or more polypeptides. Thus, the terms “protein”, “peptide” and “polypeptide” can be used interchangeably. Polypeptides may optionally be modified (e.g., glycosylated, phosphorylated, acylated, farnesylated, prenylated, sulfonated, and the like) to add functionality. Polypeptides exhibiting activity may be referred to as enzymes. It will be understood that, as a result of the degeneracy of the genetic code, a multitude of nucleotide sequences encoding a given polypeptide may be produced.

The polypeptide may be comprised in a composition. Preferably, the composition is enriched in such a polypeptide. The composition may comprise a polypeptide of the present invention as the major enzymatic component, e.g., a mono-component composition. Alternatively, the composition may comprise multiple enzymatic activities. The polypeptide compositions may be prepared in accordance with methods known in the art and may be in the form of a liquid or a dry composition. For instance, the polypeptide composition may be in the form of a granulate or a microgranulate. The polypeptide to be included in the composition may be stabilized in accordance with methods known in the art. The dosage of the polypeptide composition of the invention and other conditions under which the composition is used depend on the ultimate use of the composition.

The term “polypeptide fragment” is defined herein as a polypeptide having one or more amino acids deleted from the amino and/or carboxyl terminus of the parent polypeptide or a homologous sequence thereof.

A MM-oxidase polypeptide of the invention may be in isolated form or in substantially pure form.

The term “isolated polypeptide” as used herein means a polypeptide that is removed from at least one component, e.g. other polypeptide material, with which it is naturally associated. Thus, an isolated polypeptide may contain at most 10%, at most 8%, more preferably at most 6%, more preferably at most 5%, more preferably at most 4%, more preferably at most 3%, even more preferably at most 2%, even more preferably at most 1% and most preferably at most 0.5% as determined by SDS-PAGE of other polypeptide material with which it is natively associated. The isolated polypeptide may be free of any other impurities. The isolated polypeptide may be at least 50% pure, e.g., at least 60% pure, at least 70% pure, at least 75% pure, at least 80% pure, at least 85% pure, at least 80% pure, at least 90% pure, or at least 95% pure, 96%, 97%, 98%, 99%, 99.5%, 99.9% as determined by SDS-PAGE or any other analytical method suitable for this purpose and known to the person skilled in the art.

The term “substantially pure” with regard to polypeptides refers to a polypeptide preparation which contains at the most 50% by weight of other polypeptide material. The polypeptides disclosed herein are preferably in a substantially pure form. In particular, it is preferred that the polypeptides disclosed herein are in “essentially pure form”, i.e. that the polypeptide preparation is essentially free of other polypeptide material. Optionally, the polypeptide may also be essentially free of non-polypeptide material such as nucleic acids, lipids, media components, and the like. Herein, the term “substantially pure polypeptide” is synonymous with the terms “isolated polypeptide” and “polypeptide in isolated form”. The term “substantially pure” with regard to polynucleotide refers to a polynucleotide preparation which contains at the most 50% by weight of other polynucleotide material. The polynucleotides disclosed herein are preferably in a substantially pure form. In particular, it is preferred that the polynucleotide disclosed herein are in “essentially pure form”, i.e. that the polynucleotide preparation is essentially free of other polynucleotide material. Optionally, the polynucleotide may also be essentially free of non-polynucleotide material such as polypeptides, lipids, media components, and the like. Herein, the term “substantially pure polynucleotide” is synonymous with the terms “isolated polynucleotide” and “polynucleotide in isolated form”.

A polypeptide of the invention may be “naturally-occurring”. The term “naturally-occurring” as used herein refers to processes, events, or things that occur in their relevant form in nature. By contrast, “not naturally-occurring” refers to processes, events, or things whose existence or form involves the hand of man. Generally, the term “naturally-occurring” with regard to polypeptides or nucleic acids can be used interchangeable with the term “wild-type” or “native”. It refers to polypeptide or nucleic acids encoding a polypeptide, having an amino acid sequence or polynucleotide sequence, respectively, identical to that found in nature. Naturally occurring polypeptides include native polypeptides, such as those polypeptides naturally expressed or found in a particular host. Naturally occurring polynucleotides include native polynucleotides such as those polynucleotides naturally found in the genome of a particular host. Additionally, a sequence that is wild-type or naturally-occurring may refer to a sequence from which a variant or a synthetic sequence is derived.

The invention also provides a polynucleotide coding for the polypeptide of the invention. Such a polynucleotide may be selected from the group consisting of:
(a) a polynucleotide sequence comprising SEQ ID NO: 1 or SEQ ID NO: 3 or comprising a polynucleotide sequence having at least 50% sequence identity with the polynucleotide sequence of SEQ ID NO: 1 or SEQ ID NO: 3;
(b) a polynucleotide sequence which is degenerate as a result of the degeneracy of the genetic code to a polynucleotide sequence as defined in any one of (a) or (b); or
(c) a polynucleotide sequence which is the reverse complement of a nucleotide sequence as defined in (a), (b) or (c).

Such a polynucleotide may have a sequence identity of at least 60%, preferably at least 70%, more preferably at least 80%, most preferably at-least 90%, most preferably at least 93%, most preferably at least about 95%, most preferably at least about 96%, most preferably at least about 97%, even most preferably at least about 98%, and even more preferred at least 99% to SEQ ID NO: 1 or 3.

The term “reverse complement” can be used interchangeably with the terms complementary strand” and “complement”. The reverse complement of a nucleic acid strand can be the reverse complement of a coding strand or the reverse complement of a non-coding strand. When referring to double-stranded nucleic acids, the reverse complement of a nucleic acid encoding a polypeptide refers to the reverse complementary strand of the strand encoding the amino acid sequence or to any nucleic acid molecule containing the same.

The term “degenerate” in relation to a polynucleotide or nucleic acid sequence of the invention denotes a sequence of nucleic acids that includes one or more degenerate codons (as compared to a reference nucleic acid molecule that encodes a polypeptide). Degenerate codons contain different triplets of nucleic acids, but encode the same amino acid residue (i.e., GAU and GAC triplets each encode Asp). The codon degeneracy refers to the nature of the genetic code permitting variation of the nucleic acid sequence without affecting the amino acid sequence of an encoded polypeptide. The skilled artisan is well aware of the “codon-bias” exhibited by a specific host cell in usage of nucleic acid codons to specify a given amino acid.

The polynucleotide of the invention may be in the form of a cDNA. A “cDNA” (complementary DNA) is defined herein as a DNA molecule which can be prepared by reverse transcription from a mRNA molecule. In prokaryotes the mRNA molecule is obtained from the transcription of the genomic DNA of a gene present in a cell. In eukaryotic cells genes contain both exons, i.e. coding sequences, and introns, i.e. intervening sequences located between the exons. Therefore in eukaryotic cell the initial, primary RNA obtained from transcription of the genomic DNA of a gene is processed through a series of steps before appearing as mRNA. These steps include the removal of intron sequences by a process called splicing. cDNA derived from mRNA only contains coding sequences and can be directly translated into the corresponding polypeptide product.

A polynucleotide of the invention may be in “isolated” form. An “isolated polynucleotide” or “isolated nucleic acid” is a polynucleotide removed from other polynucleotides with which it is naturally associated. Thus, an isolated polynucleotide may contain at most 10%, at most 8%, more preferably at most 6%, more preferably at most 5%, more preferably at most 4%, more preferably at most 3%, even more preferably at most 2%, even more preferably at most 1% and most preferably at most 0.5% by weight of other polynucleotide material with which it is naturally associated. The isolated polynucleotide may be free of any other impurities. The isolated polynucleotide may be at least 50% pure, e.g., at least 60% pure, at least 70% pure, at least 75% pure, at least 80% pure, at least 85% pure, at least 90% pure, or at least 95%, 96%, 97%, 98%, 99%, 99.5%, 99.9% pure by weight.

The invention provides a nucleic acid construct which comprises a polynucleotide of the invention. Such a nucleic acid construct may be an expression vector, wherein the polynucleotide sequence of the invention is operably linked to at least one control sequence for the expression of the polynucleotide sequence in a host cell.

The term “nucleic acid construct” is herein referred to as a nucleic acid molecule, either single- or double-stranded, which is isolated from a naturally occurring gene or which has been modified to contain segments of nucleic acid which are combined and juxtaposed in a manner which would not otherwise exist in nature. The term nucleic acid construct is synonymous with the term “expression cassette” when the nucleic acid construct contains all the control sequences required for expression of a coding sequence, wherein said control sequences are operably linked to said coding sequence.

The term “operably linked” as used herein refers to two or more nucleic acid sequence elements that are physically linked and are in a functional relationship with each other. For instance, a promoter is operably linked to a coding sequence if the promoter is able to initiate or regulate the transcription or expression of a coding sequence, in which case the coding sequence should be understood as being “under the control of” the promoter. Generally, when two nucleic acid sequences are operably linked, they will be in the same orientation and usually also in the same reading frame. They usually will be essentially contiguous, although this may not be required.

The term “control sequence” can be used interchangeably with the term “expression-regulating nucleic acid sequence”. The term as used herein refers to nucleic acid sequences necessary for and/or affecting the expression of an operably linked coding sequence in a particular host organism or in vitro. When two nucleic acid sequences are operably linked, they usually will be in the same orientation and also in the same reading frame. They usually will be essentially contiguous, although this may not be required. The expression-regulating nucleic acid sequences, such as inter alia appropriate transcription initiation, termination, promoter, leader, signal peptide, propeptide, prepropeptide, or enhancer sequences; Shine-Delgarno sequence, repressor or activator sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion, can be any nucleic acid sequence showing activity in the host organism of choice and can be derived from genes encoding proteins, which are either homologous or heterologous to the host organism. Each control sequence may be native or foreign to the nucleic acid sequence encoding the polypeptide. When desired, the control sequence may be provided with linkers for the purpose of introducing specific restriction sites facilitating ligation of the control sequences with the coding region of the nucleic acid sequence encoding a polypeptide. Control sequences may be optimized to their specific purpose.

The invention also provides an expression vector comprising a polynucleotide coding for a polypeptide of the invention, operably linked to the appropriate control sequences (such as a promoter, and transcriptional and translational stop signals) for expression and/or translation in vitro, or in the host cell of the polynucleotide.

The expression vector of the invention may be any vector (e.g., a plasmid or virus), which can be conveniently subjected to recombinant DNA procedures and can bring about the expression of the polynucleotide. The choice of the vector will typically depend on the compatibility of the vector with the cell into which the vector is to be introduced. The vectors may be linear or closed circular plasmids. The vector may be an autonomously replicating vector, i.e., a vector, which exists as an extra-chromosomal entity, the replication of which is independent of chromosomal replication, e.g., a plasmid, an extra-chromosomal element, a mini-chromosome, or an artificial chromosome.

Alternatively, the vector may be one which, when introduced into the host cell, is integrated into the genome and replicated together with the chromosome(s) into which it has been integrated. The integrative cloning vector may integrate at random or at a predetermined target locus in the chromosomes of the host cell.

The vector system may be a single vector or plasmid or two or more vectors or plasmids, which together contain the total DNA to be introduced into the genome of the host cell, or a transposon.

A vector of the invention preferably contains one or more selectable markers, which permit easy selection of transformed cells. A selectable marker is a gene which allow for selection of cells transformed with such gene and which provides for biocide or viral resistance, resistance to heavy metals, prototrophy to auxotrophs, and the like. The selectable marker may be introduced into the cell on the expression vector as the expression cassette or may be introduced on a separate expression vector.

Preferred selectable markers include, but are not limited to, those which confer resistance to drugs or which complement a defect in the host cell. They include e.g. versatile marker genes that can be used for transformation of most filamentous fungi and yeasts such as acetamidase genes or cDNAs (the amdS, niaD, facA genes or cDNAs from A. nidulans, A. oryzae or A. niger), or genes providing resistance to antibiotics like G418, hygromycin, bleomycin, kanamycin, methotrexate, phleomycin orbenomyl resistance (benA). Alternatively, specific selection markers can be used such as auxotrophic markers which require corresponding mutant host strains: e.g. URA3 (from S. cerevisiae or analogous genes from other yeasts), pyrG or pyrA (from A. nidulans or A. niger), argB (from A. nidulans or A. niger) or trpC. In a preferred embodiment the selection marker is deleted from the transformed host cell after introduction of the expression construct so as to obtain transformed host cells which are free of selection marker genes.

Other markers include ATP synthetase, subunit 9 (oliC), orotidine-5′-phosphatedecarboxylase (pvrA), the bacterial G418 resistance gene (this may also be used in yeast, but not in fungi), the ampicillin resistance gene (E. coli), the neomycin resistance gene (Bacillus) and the E. coli uidA gene, coding for β-glucuronidase (GUS).

The term selectable marker extends to a marker gene used for screening, i.e. marker gene that, once introduced into a host cell confers to the cell a visible phenotype and causes the cell look different. An example of marker for screening is the gene coding for the Green fluorescent protein which causes cells glow green under UV light.

The invention further provides a host cell comprising a polynucleotide or nucleic acid construct of the invention. A host cell as defined herein is an organism suitable for genetic manipulation and one which may be cultured at cell densities useful for industrial production of a target product. A suitable organism may be a microorganism, for example one which may be maintained in a fermentation device. A host cell may be a host cell found in nature or a host cell derived from a parent host cell after genetic manipulation or classical mutagenesis.

A host cell may be a prokaryotic, archaebacterial or eukaryotic host cell.

A prokaryotic host cell may, but is not limited to, a bacterial host cell. An eukaryotic host cell may be, but is not limited to, a yeast, a fungus, an amoeba, an algae, an animal, an insect host cell.

An eukaryotic host cell may be a fungal host cell. “Fungi” include all species of the subdivision Eumycotina (Alexopoulos, C. J., 1962, In: Introductory Mycology, John Wiley & Sons, Inc., New York). The term fungus thus includes among others filamentous fungi and yeast.

“Filamentous fungi” are herein defined as eukaryotic microorganisms that include all filamentous forms of the subdivision Eumycotina and Oomycota (as defined by Hawksworth et al., 1995, supra). The filamentous fungi are characterized by a mycelial wall composed of chitin, cellulose, glucan, chitosan, mannan, and other complex polysaccharides. Vegetative growth is by hyphal elongation and carbon catabolism is obligatory aerobic. Filamentous fungal strains include, but are not limited to, strains of Acremonium, Aspergillus, Agaricus, Aureobasidium, Cryptococcus, Corynascus, Chrysosporium, Filibasidium, Fusarium, Humicola, Magnaporthe, Monascus, Mucor, Myceliophthora, Mortierella, Neocallimastix, Neurospora, Paecilomyces, Penicillium, Piromyces, Phanerochaete Podospora, Pycnoporus, Rhizopus, Schizophyllum, Sordaria, Talaromyces, Rasmsonia, Thermoascus, Thielavia, Tolypocladium, Trametes and Trichoderma. Preferred filamentous fungal strains that may serve as host cells belong to the species Aspergillus niger, Aspergillus oryzae, Aspergillus fumigatus, Penicillium chrysogenum, Penicillium citrinum, Acremonium chrysogenum, Trichoderma reesei, Rasamsonia emersonii (formerly known as Talaromyces emersonii), Aspergillus sojae, Chrysosporium lucknowense, Myceliophtora thermophyla. Reference host cells for the comparison of fermentation characteristics of transformed and untransformed cells, include e.g. Aspergillus niger CBS120.49, CBS 513.88, Aspergillus oryzae ATCC16868, ATCC 20423, IFO 4177, ATCC 1011, ATCC 9576, ATCC14488-14491, ATCC 11601, ATCC12892, Aspergillus fumigatus AF293 (CBS101355), P. chrysogenum CBS 455.95, Penicillium citrinum ATCC 38065, Penicillium chrysogenum P2, Acremonium chrysogenum ATCC 36225, ATCC 48272, Trichoderma reesei ATCC 26921, ATCC 56765, ATCC 26921, Aspergillus sojae ATCC11906, Chrysosporium lucknowense ATCC44006 and derivatives of all of these strains. Particularly preferred as filamentous fungal host cell are Aspergillus niger CBS 513.88 and derivatives thereof.

A eukaryotic host cell may be a yeast cell. Preferred yeast host cells may be selected from the genera: Saccharomyces (e.g., S. cerevisiae, S. bayanus, S. pastorianus, S. carlsbergensis), Kluyveromyces, Candida (e.g., C. revkaufi, C. pulcherrima, C. tropicalis, C. utilis), Pichia (e.g., P. pastoris), Schizosaccharomyces, Hansenula, Kloeckera, Schwanniomyces, and Yarrowia (e.g., Y. lipolytica (formerly classified as Candida lipolytica)).

Prokaryotic host cells may be bacterial host cells. Bacterial host cell may be Gram negative or Gram positive bacteria. Examples of bacteria include, but are not limited to, bacteria belonging to the genus Bacillus (e.g., Bacillus alkalophilus, Bacillus amyloliquefaciens, Bacillus brevis, Bacillus circulans, Bacillus clausii, Bacillus coagulans, Bacillus firmus, B. halodurans, Bacillus lautus, Bacillus lentus, Bacillus licheniformis, Bacillus megaterium, Bacillus puntis, Bacillus pumilus, Bacillus stearothermophilus, Bacillus subtilis, or Bacillus thuringiensis), Acinetobacter, Nocardia, Xanthobacter, Escherichia (e.g., E. coli (e.g., strains DH 10B, Stbl2, DH5-alpha, DB3, DB3.1), DB4, DB5, JDP682 and ccdA-over (e.g., U.S. application Ser. No. 09/518,188)), TOP10F′, TOP10, DH10B, DH5a, HB101, W3110, BL21(DE3), BL21 (DE3)pLysS and Escherichia coli K-12 strains, e.g. DH1, HB101, RV308, RR1, W3110, C600). Streptomyces (e.g., Streptomyces lividans or Streptomyces murinus) Erwinia, Klebsiella, Serratia (e.g., S. marcessans), Pseudomonas (e.g., P. aeruginosa), Salmonella (e.g., S. typhimurium, S. typhi). Bacteria also include, but are not limited to, photosynthetic bacteria (e.g., green non-sulfur bacteria (e.g., Choroflexus bacteria (e.g., C. aurantiacus), Chloronema (e.g., C. gigateum)), green sulfur bacteria (e.g., Chlorobium bacteria (e.g., C. limicola), Pelodictyon (e.g., P. luteolum), purple sulfur bacteria (e.g., Chromatium (e.g., C. okenii)), and purple non-sulfur bacteria (e.g., Rhodospirillum (e.g., R. rubrum), Rhodobacter (e.g. R. sphaeroides, R. capsulatus), and Rhodomicrobium bacteria (e.g., R. vanellii)).

Host cells may be host cells from non-microbial organisms. Examples of such cells, include, but are not limited to, insect cells (e.g., Drosophila (e.g., D. melanogaster), Spodoptera (e.g., S. frugiperda Sf9 or Sf21 cells) and Trichoplusa (e.g., High-Five cells); nematode cells (e.g., C. elegans cells); avian cells; amphibian cells (e.g., Xenopus laevis cells); reptilian cells; and mammalian cells (e.g., NIH3T3, 293, CHO, COS, VERO, C127, BHK, Per-C6, Bowes melanoma and HeLa cells).

The invention further provides a method of producing a polypeptide of the invention comprising:
(a) cultivating the host cell of the invention under conditions conducive to the production of the polypeptide by the host cell; and, optionally,
(b) recovering the polypeptide.

Accordingly, overexpression of a polypeptide of the invention may be accomplished by methods well known to those skilled in the art. Both prokaryotic and eukaryotic cells suitable for expression of a polypeptide of the invention are described above.

The required overexpression in the preferred host cell is established using selected promoters and other expression regulation signals which are compatible with the host cell for which expression is designed. In a preferred embodiment, the promoter sequences may be obtained from a bacterial source, such as a Bacillus strain or Escherichia strain as described above.

Examples of suitable promoter sites include the promoter of the Bacillus lentus alkaline protease gene (aprH), the promoter of the Bacillus licheniformis alkaline protease gene (subtilisin Carlsberg gene), the promoter of the Bacillus subtilis levansucrase gene (sacB), the promoter of the Bacillus subtilis alphaamylase gene (amyF), the promoter of the Bacillus licheniformis alphaamylase gene (amyL), the promoter of the Bacillus stearothermophilus maltogenic amylase gene (amyM), the promoter of the Bacillus amyloliquefaciens alpha-amylase gene (amyQ). Yet another example is a “consensus” promoter having the sequence TTGACA for the “−35” region and TATAAT for the “−10” region. And additionally the promoter of the Bacillus licheniformis penicillinase gene (penP) and the promoters of the Bacillus subtilis xylA and xylB genes.

Alternatively, E. coli may be used as a host cell. Suitable vectors are the vectors normally used for cloning and expression and are known to the person skilled in the art, for example, the cos, tac, trp, tet, trp-tet, Ipp, lac, Ipp-lac, lacIq, T7, T5, T3, gal, trc, ara, SP6, I-PR or I-PL promoters. Examples of suitable vectors for expression in E. coli are given e.g. in table 1 in Makrides, S. C., Microbiological Reviews, Vol. 60, No. 3, (1996), 512-538. Preferably, the vector contains a promoter upstream of the cloning site containing the nucleic acid sequence encoding the MM-oxidase, which can be switched on after the host has been grown to express the corresponding polypeptide having MM-oxidase activity. Promoters, which can be switched on and off are known to the person skilled in the art and are for example the lac promoter, the araBAD promoter, the T7 promoter, the trc promoter, the tac promoter and the trp promoter. Particularly useful in the framework of the invention are for example the vectors as described in WO 00/66751, e.g. pKAFssECtrp or pKAFssECaro without the insert, the penicillin G acylase gene.

The choice of the vector can sometimes depend on the choice of the host and vice versa. If e.g. a vector with the araBAD promoter is being used, an E. coli host strain that is unable to break down the arabinose inducer (ara-), is strongly preferred.

A nucleic acid sequence encoding a MM-oxidase can be integrated into the genome of a host cell, which does not normally contain a nucleic acid sequence according to the invention and be (over)expressed. This can be done according to methods known to the person skilled in the art.

Expression of MM-oxidase of the invention may be carried out by culturing host cells as described herein. After fermentation, the host cells can be harvested by either centrifugation or by filtration. The cells are then resuspended and lysed by a well known methods such as incubating with lysozyme or by using a French press. In the case that E. coli is used a host cell, the cells may preferably be lysed by freezing the cells, followed by a lysozyme treatment.

The lysed cell mass is then again filtered or centrifuged to obtain the MM-oxidase solubilised in the filtrate or supernatant. If required the MM-oxidase can be purified by any suitable chromatographic method e.g. by selective precipitation or chromatographic methods described in the existing scientific literature on MM-oxidase.

The invention thus relates to a composition, such as an oral composition, comprising a polypeptide of the invention and at least one additional ingredient.

The oral composition of the invention (which comprises any MM-oxidase, for example a polypeptide of the invention) may be in any suitable form such that it may be applied to the oral cavity. Typically, the oral composition of the invention may be a liquid or a solid composition.

The term “liquid formulation” as used herein denotes a formulation, i.e. a mixture of two or more components according to a formula, which is liquid at a temperature of at least about 2° C. or higher under atmospheric pressure. The liquid formulation may contain water or an organic liquid or a mixture thereof. An organic liquid is herewith defined as an organic substance or mixture of two or more organic substances which is liquid at a temperature of at least 2° C. or higher under atmospheric pressure. An organic substance is made of molecules which contain carbon. The liquid formulation may be stabilized by addition of one or more stabilisers to yield a stabilized liquid formulation. A stabilized liquid formulation is a liquid formulation in which the active ingredient(s) comprised therein essentially retain their chemical, physical and/or biological activity upon storage under specific conditions. For example the stabilized liquid formulation may be stable at a refrigerated temperature (2-8° C.) for at least 12 months, preferably 2 years, and more preferably 3 years; or at room temperature (23-27° C.) for at least 3 months, preferably 6 months, and more preferably 1 year.

The term “solid formulation” refers to formulation, i.e. a mixture of two or more components according to a formula, which is in the solid state rather than a liquid or gas. The solid formulation is preferably in the solid state at the temperature of 100° C. or lower. Liquids, dispersions and solutions are not encompassed by this term. Further, semi-solids such as gels, ointments, pastes and creams are neither encompassed by this term. Representative examples of solid formulations that are encompassed include capsules, tablets, pellets, granules, and powders.

Solid oral compositions of the invention in which an MM-oxidase enzyme, for example a polypeptide of the invention, may be incorporated, to provide a composition having an ability to control breath odour includes for example chewing gums (e.g., compressed gums), confections (e.g., hard boiled candy, low boiled candy and chewy candies), pet chew or biscuit, nougats, chocolates, toffees, dragees, caramels, lozenges, throat drops, pressed tablets, such as a pressed mint, capsules, edible films, nuts, foams, dentifrices such as toothpaste, toothpowders and combinations thereof.

A liquid composition of the invention may be a mouthrinse/mouthwash or a mouth spray for example or a syrup.

A chewing gum is any type of gum made from, for example, chicle, a natural latex product, or a synthetic rubber known as polyisobutylene, for example.

A toothpaste is a paste or gel dentifrice, typically used with a toothbrush as an accessory to clean and maintain the aesthetics and health of teeth.

A mouthwash or mouthrinse is any liquid product used to enhance oral hygiene which is typically used by gargling and then spat out.

Generally speaking, the oral composition of the present invention may be prepared by any means or method known to one of ordinary skill in the art. Typically, however, the oral composition is prepared by a method that comprises contacting a MM-oxidase enzyme with one or more components of the oral composition to form a mixture.

For example, the method typically includes placing the enzyme, for example in an encapsulated form, in a vessel containing the one or more components of the oral composition, or the finished oral composition but for the encapsulated enzyme (i.e., the encapsulated enzyme is added last). Generally, the method further comprises forming the mixture of components into a suitable shape for oral consumption.

Although the MM-oxidase is capable of oxidizing the H₂S formed during the oxidation of the methane thiol present (as shown in the original paper), the enzyme can also be combined with chemicals known to bind H₂S such as zinc salts, in particular organic zinc salts. Organic zinc salts suitable for use in this way in include zinc ascorbate, zinc hydrogen acetate, zinc lactate, zinc citrate, zinc gluconate and combinations thereof.

Additionally, compositions may be formulated to increase residence time in the mouth. For example, WO2007/143989 discloses chewing gums comprising a hydrophobic enzyme formulation that reduces release of enzymes from a chewing gum such that the residence time in the mouth of enzyme is increased.

Compositions may also be formulated to contain compounds that adjust the pH of the mouth to a pH that is optimal for the MM-oxidase enzyme.

An oral composition may be in the form of a dentifrice comprising MM-oxidase. Enzyme containing dentifrice compositions are well-known to the skilled person. For example, U.S. Pat. No. 4,150,113 and U.S. Pat. No. 4,178,362 disclose, respectively, an enzymatic toothpaste and an enzymatic chewable dentifrice containing glucose oxidase. Further enzyme containing dentifrices are disclosed in, for example, U.S. Pat. No. 4,537,764, U.S. Pat. No. 4,578,365, U.S. Pat. No. 4,564,519, U.S. Pat. No. 5,176,899, U.S. Pat. No. 4,564,519 and U.S. Pat. No. 4,578,265. These documents describe in detail formulation of enzymes into dentifrices, such as toothpastes, and may be applied to the formulation of MM-oxidase into dentifrice compositions.

An oral composition of the invention may be in the form of a chewing gum, which chewing gum may comprise:
a water insoluble base;
a water soluble portion;
a volatile sulphur compound breath-odour controlling amount of methyl mercaptan oxidase; and, optionally
a sweetener and/or a flavour.

The MM-oxidase in such a chewing gum can be contained in a variety of different chewing gum compositions. The chewing gum can be a number of different structures. For example, the chewing gum can be a single piece, for example, a stick, slab, or other unitary structure.

On the other hand, the chewing gum can comprise an over-coated formulation.

In this regard, if desired, the MM-oxidase can be located within a coating or shell that substantially encloses a gum center. The coating can comprise, in an embodiment, approximately 40 to about 75% of the chewing gum composition. In addition to the MM-oxidase, the coating can include a masking agent to improve the taste of the coating containing the MM-oxidase.

A variety of masking agents can be utilized including: sucralose; zinc gluconate; ethyl maltol; glycine; acesulfame-k; aspartame; saccharin; fructose; xylitol; spray dried licorice root; glycerrhizine; dextrose; sodium glutonate; glucono delta-lactone; ethyl vanillin; vanillin; normal and high potency sweeteners; and a variety of appropriate flavors. A sufficient masking agent may be used to mask the taste of the MM-oxidase enzyme preparation. If desired, more than one masking agent can be used.

A variety of methods can be used for creating a coated chewing gum. For example, the coating can be applied in a three phase operation to a chewing gum center.

In the first phase, a crude coating of an alternate application of syrup and powder is applied to the center. This is followed by a second phase called the finishing coating in which a fine powder and longer tumbling is used to produce a smooth finish. Finally, a shellacking and polishing third phase is performed to provide a high sheen, smooth finish. If desired, the second and third phases can be eliminated. The coating can surround a variety of different types of gum center compositions as set forth below.

In another embodiment of the present invention, a compressible excipient is tableted and then coated with a chewing gum product including the MM-oxidase. The tableted excipient can comprise, by way of example and not limitation, dextrose, sucrose, or other saccharides, sorbitol, mannitol, isomalitol, other compressible sugar alcohols or combinations thereof. The tableted compressible excipient is substantially surrounded by a gum coating. The coating includes the MM-oxidase and, in an embodiment, comprises at least 50% by weight of the product. Additionally, the coating can include a masking agent and the chewing gum.

Referring now to the chewing gum of the present invention, the chewing gums can are preferably low moisture, sugar or sugarless, wax containing or wax free, low calorie (via high base or low calorie bulking agents), and/or may contain other dental and/or medicinal agents.

In general, a chewing gum typically comprises a water-soluble bulk portion, a water-insoluble chewable gum base portion, and, optionally, a flavouring and/or sweetening agent. The water-soluble portion dissipates with a portion of the flavoring agent over a period of time during chewing. The gum base portion is retained in the mouth throughout the chew. The term chewing gum refers to both a chewing and bubble gum in its general sense.

The insoluble gum base generally comprises elastomers, resins, fats and oils, softeners and inorganic fillers. The gum base may or may not include wax. The insoluble gum base can constitute approximately 5% to about 95% by weight of the chewing gum, more commonly the gum base comprises 10% to about 50% of the gum, and in some preferred embodiments approximately 15% to about 35%, by weight, of the chewing gum.

The chewing gum base of the present invention may contains about 20% to about 60% by weight synthetic elastomer, about 0% to about 30% by weight natural elastomer, about 5% to about 55% by weight elastomer plasticizer, about 4% to about 35% by weight filler, about 5% to about 35% by weight softener, and optional minor amounts (about 1% or less by weight) of miscellaneous ingredients such as colorants, antioxidants, etc.

Elastomers provide the rubbery, cohesive nature of the gum, which varies depending on this ingredient's chemical structure and how it is compounded with other ingredients. Synthetic elastomers may include, but are not limited to, polyisobutylene, isobutylene-isoprene copolymer (butyl rubber), styrene-butadiene, copolymers having styrene-butadiene ratios of about 1:3 to about 3:1, polyvinyl acetate, vinyl acetatevinyl laurate copolymer having a vinyl laurate content of about 5% to about 50% by weight of the copolymer, and combinations thereof.

Natural elastomers may include natural rubber such as smoked or liquid latex and guayule as well as natural gums such as jelutong, lechi caspi, perillo, sorva, massaranduba balata, massaranduba chocolate, nispero, rosindinha, chicle, gutta hang kang, and combinations thereof. The preferred synthetic elastomer and natural elastomer concentrations vary depending on whether the chewing gum in which the base is used is adhesive or conventional, bubble gum or regular gum. Preferred natural elastomers include jelutong, chicle, sorva and massaranduba balata.

Elastomer plasticizers may include, but are not limited to, rosin esters such as glycerol esters of rosin, methyl esters of rosin, pentaerythritol esters of rosin; terpene resins derived from alpha-pinene, beta-pinene, and/or d-limonen; and any suitable combinations of the foregoing. The resin tackifiers regulate the cohesiveness and tackiness of the final gums. The preferred elastomer plasticizers will also vary depending on the specific application, and on the type of elastomer which is used.

Fillers/texturizers may include magnesium and calcium carbonate, ground limestone, silicate types such as magnesium and aluminum silicate, clay, alumina, talc, titanium oxide, mono-, di- and tri-calcium phosphate, cellulose polymers, such as wood, and combinations thereof. Fillers modify the texture of the gum base. The fillers can also be organic powders such as polyethylene, oat fiber, wood fiber, apple fiber, zein, gluten, gliadin, casein, and the like. MM-oxidase powder can be added as a filler during base making to achieve better encapsulation which may result in longer release of MM-oxidase.

Softeners/emulsifiers may include tallow, hydrogenated tallow, hydrogenated and partially hydrogenated vegetable oils, cocoa butter, glycerol monostearate, glycerol triacetate, lecithin, non-hydrogenated, partially hydrogenated and fully hydrogenated mono-, di- and tri-glycerides from cottonseed, soybean, palm, palm kernel, coconut, and safflower sources, and other medium chain triglycerides, acetylated monoglycerides, fatty acids (e.g. stearic, plasmatic, oleic and linoleic acids), and combinations thereof.

Such softeners/emulsifiers modify the texture of the gum base by introducing sharp melting transition during chewing.

Colorants and whiteners may include FD & C-type dyes and lakes, fruit and vegetable extracts, titanium dioxide, and combinations thereof. Colorants impart characteristics and remove or mask undesired characteristics in the chewing gum formulation.

The gum base may or may not include wax. An example of a wax-free gum base is disclosed in U.S. Pat. No. 5,286,500, the disclosure of which is incorporated herein by reference. Waxes aid in the curing of gum bases and in improving shelf life and texture of the final gum product. Wax crystal also improves the release of flavor from the final product.

Such gum bases are typically prepared by adding an amount of the elastomer, resin tackifier or softer, and filler to a pre-heated sigma blade mixer having a temperature of from about 10° C. to about 115° C. The initial amounts of ingredients comprising the initial mass of the insoluble gum base may be determined by the working capacity of the mixing kettle in order to attain a proper consistency and by the degree of compounding desired to break down and soften the elastomer. The longer the period of time compounding and use of lower molecular weight or softening point gum base ingredients, a lower viscosity and firmness will result in the final gum base.

In addition to a water insoluble gum base portion, a typical chewing gum composition includes a water soluble bulk portion which may optionally contain one or more flavoring agents and/or sweetening agents.

Thus, the water soluble portion can include bulk sweeteners, high intensity sweeteners, flavoring agents, softeners, emulsifiers, colors, acidulants, fillers, antioxidants, medicaments, and other components that provide desired attributes.

Softeners are added to the chewing gum in order to optimize the chewability and mouth feel of the gum. The softeners, which are also known as plasticizers, and plasticizing agents, generally constitute between approximately 0.5% to about 25% by weight of the chewing gum. The softeners may include glycerin, lecithin, and combinations thereof. Aqueous sweetener solutions such as those containing sorbitol, hydrogenated starch hydrolysates, corn syrup and combinations thereof, may also be used as softeners and binding agents in chewing gum.

Bulk sweeteners include both sugar and sugarless components. Bulk sweeteners typically constitute about 5% to about 95% by weight of the chewing gum, more typically, about 20% to about 80% by weight, and more commonly, about 30% to about 60% by weight of the gum. Sugar sweeteners generally include saccharide-containing components commonly known in the chewing gum art, including but not limited to, sucrose, dextrose, maltose, dextrin, dried invert sugar, fructose, levulose, glactose, corn syrup solids, and the like, alone or in combination. Sugarless sweeteners include, but are not limited to, sugar alcohols such as sorbitol, mannitol, xylitol, hydrogenated starch hydrolysates, maltitol, and the like, alone or in combination.

High intensity artificial sweeteners can also be used, alone or in combination, with the above. Preferred sweeteners include, but are not limited to, sucralose, aspartame, salts of acesulfame, altitame, saccharin and its salts, cyclamic acid and its salts, glycerrhizinate, dihydrochalcones, thaumatin, monellin, and the like, alone or in combination. The range of these sweeteners in chewing gum formulations typically can range from about 0.02 to about 0.10 weight percent for alitame, thaumatin and dihydrochalcones, and from about 0.1 to about 0.2 weight percent for aspartame, sucralose, acesulfam and saccharin.

In order to provide longer lasting sweetness and flavor perception, it may be desirable to encapsulate or otherwise control the release of at least a portion of the artificial sweetener. Techniques such as wet granulation, wax granulation, spray drying, spray chilling, fluid bed coating, coacervation, and fiber extension may be used to achieve the desired release characteristics.

Combinations of sugar and/or sugarless sweeteners may be used in the chewing gum. Additionally, the softener may also provide additional sweetness such as with aqueous sugar or alditol solutions.

If a low calorie gum is desired, a low caloric bulking agent can be used. Examples of low caloric bulking agents include, but are not limited to: polydextrose; Raftilose, Raftilin; Fructooligosaccharides (NutraFlora); Palatinose oligosaccharide; Guar Gum Hydrolysate (Sun Fiber); or indigestible dextrin (Fibersol). However, other low calorie bulking agents can also be used.

A variety of flavoring agents can be used, if desired. Flavoring agents like colorants are useful in chewing gum compositions to impart characteristics and to remove or mask undesired characteristics. In doing so, the flavoring agent increases the contact time of the chewing gum composition of the present invention in the oral cavity. In doing so, the chewing gum composition enhances the availability of the MM-oxidase component and prolongs the enzymatic effect by gradually releasing the enzyme from the chewing gum composition.

The flavor can be used in amounts of about 0.1 to about 15 weight percent of the gum, and preferably, about 0.2% to about 5% by weight. Flavoring agents may include essential oils, synthetic flavors or mixtures thereof including, but not limited to, oils derived from plants and fruits such as citrus oils, fruit essences, peppermint oil, spearmint oil, other mint oils, clove oil, oil of wintergreen, anise and the like. Artificial flavoring agents and components may also be used. Natural and artificial flavoring agents such as cocoa powder and heat-modified amino acids can be used as a flavoring agent within the present invention, and may be combined in any sensorially acceptable fashion.

The flavoring agent may also include a cooling agent to enhance the flavor and perceived breath freshening of the product. In addition to menthol, cooling agents may include, for example, ethyl p-menthane carboxamide, N-2,3-trimethyl-2-isopropylbutanamide, menthyl glutarate, menthyl succinate, menthol PC carbonate, menthol EC carbonate, menthyl lactate, menthone glyceryl ketal, menthol glyceryl ether, N-tertbutyl-p-menthane-3-carboxamide, p-menthane-3-carboxylic acid glycerol ester, methyl-2-isopropyl-bicycle (2.2.1), heptane-2-carboxamide, menthol methyl ether and combinations thereof. The chewing gum of the present invention may also optionally include other breath freshening or anti-microbial ingredients, including anti-microbial essential oils and flavor components, such as peppermint, methyl salicylate, thymol, eucalyptol, cinnamic aldehyde, polyphosphate, pyrophosphate and combinations thereof, may also be used.

The chewing gum composition of the present invention can be made utilizing manufacturing procedures known within the chewing gum arts. In general, chewing gum is manufactured by sequentially adding the various chewing gum ingredients to a commercially available mixer known in the art.

The MM-oxidase may generally be added at any time during the manufacturing process but, preferably, is added near the end of mixing in order to minimize exposure of the enzyme to heat that could possible denature the enzyme. After the initial ingredients have been thoroughly mixed, the gum mass is discharged from the mixer and shaped into the desired form such as by rolling into sheets and cutting into sticks, extruded into chunks or casting into pellets or balls, which may be then coated or panned.

Generally, the ingredients are mixed by first melting the gum base and adding it to the running mixer. The base may also be melted in the mixer itself. Color or emulsifiers may also be added at this time. A softener such as glycerin may also be added at this time, along with syrup and a portion of the bulking agent/sweetener.

Further portions of the bulking agent/sweetener may then be added to the mixer thereafter. A flavoring agent is typically added with the final portion of the bulking agent/sweetener. A high-intensity sweetener is preferably added after the final portion of the bulking agent/sweetener and flavor has been added.

The entire mixing procedure typically takes from five to fifteen minutes, but longer mixing times may sometimes be required. Those skilled in the art will recognize that many variations of the above-described procedure may be followed.

In manufacturing the chewing gum composition of the present invention in particular, the MM-oxidase is mixed with the gum base, sweetener or sweetener mixture, and a flavoring agent. Preferably, the MM-oxidase is added as late as possible to the mix. The smaller the amount of MM-oxidase used, the more necessary it becomes to pre-blend that particular ingredient to assume uniform distribution throughout the batch of gum.

Whether a pre-blend is used or not, preferably the MM-oxidase is added as late as possible in mixing (to minimize heat exposure). Because the chewing gum composition of the present invention contains a water insoluble base, it enhances the gradual or controlled release of the medicament from the composition into the oral cavity. Thus, as the chewing gum composition of the present invention is chewed, the MM-oxidase component will gradually dissipate, along with any sweeteners and flavour, during chewing.

Again, such gradual release enhances the availability to the oral cavity.

Water activity is the relative availability of water in a substance. It is defined in the art as the vapour pressure of water divided by that of pure water at the same temperature. Therefore, pure distilled water has a water activity of exactly one. Water activity is different from moisture content (% water) in a food product. Moisture content is the total moisture, that is, the amount of bound plus free water present in the sample, whereas water activity only provides a measurement of the free moisture and is usually expressed as a_{w} or percentage Equilibrium Relative Humidity (% ERH). The water activity of a food product is the constant relative humidity of the air in direct vicinity of the food product when equilibrium between the food product and the surrounding air is established. This constant relative humidity is then called ‘% ERH’ if it is expressed on percentages (0 to 100%), or ‘water activity’ if it is expressed as values between 0 and 1.0. Methods for water activity determinations are detailed in the official methods of analysis of AOAC International (1995), Method 978.18. Finished products according to the invention should have a water activity below about 0.80, preferably below about 0.70, more preferably below about 0.60 and should be packed in such away that moisture is kept out.

The MM-oxidase component of the present invention may be first ground into fine particles to form a powder before being mixed with the gum base. The particle size of the MM-oxidase component is preferably from about 0.1 microns to about 200 microns in diameter, more preferably from about 1 micron to about 50 microns in diameter.

In addition, prior to the MM-oxidase powder being mixed with the gum base, the fine powder may be suspended into a liquid or liquid mixture, which is preferably water-insoluble. This is done to ease incorporation of the drug into the gum base and to enhance its uniform distribution throughout the overall chewing gum composition. Examples of liquid or liquid mixtures suitable for use within the present invention include, but are not limited to, an alcohol, an edible oil, glycerin, ethylene glycol, propylene glycol, triacetin, tributyrin glycerol mono- or di-stearate, acetylated mono-glyceride of coconut oil combinations thereof, and other like materials.

The MM-oxidase powder can be mixed with molten or softened gum base directly, or it can be pre-mixed with a gum base ingredient such as polyvinyl acetate, rosin esters, polyterpene, waxes, fats, and the like.

By providing a gradual release dosage form which can incorporate MM-oxidase, the chewing gum composition of the present invention offers an advantage because a greater amount of the enzyme is available to produce a beneficial effect over an extended period of time.

In order to maintain enzyme activity during production and subsequent storage of the finished product, high temperatures and prolonged exposure to high water activities should be prevented. Therefore, the active enzyme should not be added to the mix of ingredients used to prepare the chewable composition until this composition is cooled down and has reached the lowest possible temperature required for further processing, e.g. cutting the mass into suitable serving sizes. A similar approach should be followed in case the active enzyme is applied not in the chewable mass but in a coating applied later on. Also in the latter case heating should be kept to the minimum. Typically heating in aqueous surroundings should not exceed periods of about 30 minutes, preferably not more than about 20 minutes, more preferably not more than about 10 minutes, even more preferably not more than about 5 minutes and temperatures may typically be kept below about 60° C., preferably below about 50° C., such as below about 40° C., even more preferably below about 30° C.

The MM-oxidase enzymes used in the invention may not be optimally efficacious in oral compositions, because of their lack of stability, particularly their lack of thermal stability for use in high temperature processes (e.g., extruded gum processes).

Accordingly, an oral composition containing a coated or encapsulated MM-oxidase enzyme may be effective in the control of breath odour and may be used in low temperature, low moisture content compositions such as compressed mint and gum formulations, as well as others types of solid oral formulation.

A coated or encapsulated MM-oxidase enzymes may be more efficacious for controlling breath odour while also exhibiting improved stability and compatibility as compared to non-coated or non-encapsulated protease enzymes. More particularly, the coated or encapsulated MM-oxidase enzymes may exhibit improved stability under and compatibility with conditions typically used for the preparation of granular and/or low moisture content oral compositions of which they are a part.

Thus, a coated or encapsulated MM-oxidase may be incorporated into an oral composition that is prepared under relatively mild conditions that limit or prevent denaturing of the enzyme. In particular, the encapsulated MM-oxidase may be incorporated into oral compositions that are prepared in a manner such that the temperature of the mixture of one or more ingredients of the oral composition before and/or after introduction of the encapsulated enzyme thereto is sufficiently low, such that denaturing of the enzyme is substantially limited, if not prevented.

Additionally, the encapsulated MM-oxidase may be prepared in a manner such that the moisture content therein is sufficiently low for use in the desired oral composition. Typically, the moisture content of the encapsulated enzyme is less than about 6 wt %, less than about 4 wt %, less than about 2 wt %, or even less than about 1 wt %. For example, in certain embodiments in which the encapsulated enzyme is prepared by spray drying, the moisture content of the encapsulated enzyme may be about 1.5 wt %.

In this regard it is to be noted that the process conditions (e.g., temperature, pH, moisture, etc.) for a given oral composition and encapsulated enzyme will be, at least in part, a function of the enzyme to be used, and the desired moisture content of the oral composition in which it is to be incorporated. Accordingly, an acceptable combination of encapsulated enzyme and oral composition (e.g., oral composition components, process conditions, etc.) may be determined using means known in the art.

In accordance with the present invention, the MM-oxidase may be coated (i.e., encapsulated) in a one-step or multi-step process using one or more coating or encapsulation methods generally known in the art.

In particular, the MM-oxidase may be coated using a method, and a coating, generally known in the art, and as further detailed herein, in order to obtain a coated enzyme having improved processing capabilities, release properties, sensory properties, and/or stability. Coating or encapsulation methods known in the art, which are suitable for use in accordance with the present invention and which may be used alone or in combination, include for example spray drying, spray cooling, spray chilling, freeze-drying (i.e., lyophilizing), extrusion processes, coacervation, molecular inclusion, fluid bed coating, granulation, agglomeration, roll compaction, and absorbing the enzyme onto a support. Methods for coating or encapsulating an enzyme are well-known in the art and are described in WO2007/140286.

It is to be understood that reference to an encapsulated or coated protease enzyme herein generally refers to an enzyme that is at least partially coated or encapsulated by a coating composition (e.g., encapsulant). In one particular embodiment, substantially all the enzyme surface is coated by the encapsulant. In this regard it is to be further noted that, in those embodiments wherein the enzyme is not fully coated or encapsulated with a coating composition or encapsulant, a portion of the non-coated surface of the enzyme may be in direct contact with other ingredients or components of the oral composition including, for example, the gum base of a chewing gum composition. In such instances, it is to be understood that, in accordance with the present invention, this interaction or contact between the non-coated surface of the enzyme and other ingredients or components of the oral composition does not represent “encapsulation.”

It is to be further noted that, in addition to activity, the method of coating or encapsulation, and/or the steps or conditions used therein, may be controlled in order to optimize the size and/or control the release of the resulting encapsulated or coated enzyme. Accordingly, the particle size of the coated or encapsulated enzyme may, for example, be optimized for use in a chewing gum composition.

Typically, however, the particle size of the coated or encapsulated MM-oxidase may be at least about 10 microns, at least about 20 microns, or at least about 30 microns. In various embodiments including, for example, those in which the encapsulated enzyme is prepared by spray drying, a substantial portion of the encapsulated enzyme (e.g., at least about 90 wt %, or from 95 wt % to 100 wt %), exhibits a particle size of less than about 250 microns, less than about 200 microns, less than about 150 micron, or less than about 100 microns. Typically, the encapsulated protease enzyme exhibits a particle size of less than about 60 microns, less than 50 microns, or less than about 40 microns, the size for example falling within the range of about 10 to about 60 microns, about 20 to about 50 microns, or about 30 to about 40 microns. It is to be again further noted, however, that the particle size may be other than noted herein, without departing from the scope of the present invention.

Generally, regardless of the methods used to prepare the encapsulated enzyme, the encapsulated enzyme may typically have an enzyme concentration or, loading, of at least about 5 wt %, at least about 10 wt %, at least about 15 wt %, at least about 20 wt %, at least about 30 wt %, at least about 40 wt %, at least about 50 wt %, at least about 75 wt %, at least about 85 wt %, at least about 95 wt % (of the encapsulated MM-oxidase), or more, the loading for example ranging from about 5 wt % to about 95 wt %, from about 10 wt % to about 85 wt %, from about 10 wt % to about 50 wt %, from about 15 wt % to about 50 wt %, from about 25 wt % to about 40 wt %, or from about 20 wt % to about 40 wt % (of the encapsulated MM-oxidase).

In terms of activity, the amount of MM-oxidase present in an oral composition of the invention is an amount such that the oral composition is capable of reducing the organoleptic score to about 1 or below using a 0-5 scale (on application to a host). Alternatively, the amount of MM-oxidase present in an oral composition of the invention is an amount that may reduce the level of VSC, such as MM, to about 110 parts per billion or below as determined with a portable sulphide monitor (Halimeter®)—again on application to a host. Organoleptic scoring and the use of a portable sulphide monitor are described in Tangerman and Winkel (J Clin. Perodontol. 34, 748-755, 2007).

Alternatively, in terms of activity, the amount of MM-oxidase present in an oral composition of the invention is an amount capable of reducing the level of VSC, such as MM, by at least about 10%, at least about 20%, at least about 30%, at least about 40% at least about 50%, at least about 60%, at least about 70%, at least about 80%, at least 90%, at least about 95% or at least about 99% after the oral composition of the invention is applied to a host.

The organoleptic score, VSC amount of VSC reduction may be determined at, for example about 10 minutes, about 20 minutes, about 30 minutes, about 40 minutes, about 50 minutes, about 1 hour or longer after the oral composition is applied to the host, such as to the mouth cavity.

An oral composition according to any one of the preceding claims which does not comprise an anti-bacterial component. That is to say, the oral composition of the invention typically may not comprise a component having anti-bacterial activity.

The invention further relates to a method for controlling breath-odour. Controlling breath odour indicates that breath odour, such as mouth breath odour, will be improved. Typically this will be as a result of the reduction of one or more VSC, in particular methyl mercaptan. The reduction may be apparent organoleptically or chemically.

In such a method, the amount of one or more VSCs, such as methyl mercaptan in breath, for example mouth breath may be reduced by at least about 10%, at least about 20%, at least about 30%, at least about 40% at least about 50%, at least about 60%, at least about 70%, at least about 80%, at least 90%, at least about 95% or at least about 99% after the oral composition of the invention is applied to the oral cavity. Such reduction of MM may be determined at, for example about 10 minutes, about 20 minutes, about 30 minutes, about 40 minutes, about 50 minutes, about 1 hour or longer after the oral composition is applied to the mouth cavity. Oral compositions having a greater effect in terms of VSC reduction at shorter times after applications are preferred.

Typically, the organoleptic score is reduced to about 1 or below using a 0-5 scale and/or the level of VSC, such as MM, is reduced to about 110 parts per billion or below as determined with a portable sulphide monitor (Halimeter®).

Such a method comprises:
providing an oral composition as described herein; and
applying the composition to the oral cavity. Thus, the invention provides an oral composition which is applied to a host, for example to the human or animal body, in particular to the oral cavity of a host, such as a human or non-human animal. The invention also provides a method of controlling bad breath in a host, which method comprises the step of administering to the host an effective amount of an oral composition of the invention, typically to the oral cavity. An effective amount of a composition of the invention may be given to a host in need thereof.

Where the composition is a chewing gum, the method may comprise chewing the chewing gum to cause the methyl mercaptan oxidase to be released from the chewing gum into the oral cavity.

Accordingly, the invention provides an oral composition as described herein for controlling breath-odour.

The invention also provides use of an oral composition as described herein for use in the manufacture of a medicament for use in controlling breath-odour.

The VSCs most responsible for halitosis are also potentially damaging to the tissues in the mouth, and can lead to periodontitis (inflammation of the gums and ligaments supporting the teeth). Accordingly, the invention provides an oral composition comprising methyl mercaptan oxidase of the treatment of periodontitis or gingivitis. Thus, the invention provides an oral composition which is applied to a host, for example to the human or animal body, in particular to the oral cavity of a host, such as a human or non-human animal. The invention also provides a method of treating periodontitis or gingivitis in a host, which method comprises the step of administering to the host an effective amount of an oral composition of the invention, typically to the oral cavity. An effective amount of a composition of the invention may be given to a host in need thereof.

The terms “sequence identity” of “sequence homology” are used interchangeably herein. For the purpose of this invention, it is defined here that in order to determine the percentage of sequence homology or sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/based or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percent sequence identity between two amino acid sequences or between two nucleotide sequences may be determined using the Needleman and Wunsch algorithm for the alignment of two sequences. (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, P. Longden, I. and Bleasby, A. Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: Number of corresponding positions in the alignment showing an identical amino acid or identical nucleotide in both sequences divided by the total length of the alignment after subtraction of the total number of gaps in the alignment. The identity defined as herein can be obtained from NEEDLE by using the NOBRIEF option and is labeled in the output of the program as “longest-identity”.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., XBLAST and NBLAST) can be used. See the homepage of the National Center for Biotechnology Information at http://www.ncbi.nlm.nih.gov/.

A reference herein to a patent document or other matter which is given as prior art is not to be taken as an admission that that document or matter was known or that the information it contains was part of the common general knowledge as at the priority date of any of the claims.

The disclosure of each reference set forth herein is incorporated herein by reference in its entirety.

The present invention is further illustrated by the following Examples.

### EXAMPLES

### Molecular Biology Techniques

Molecular biology techniques known to the skilled person are performed as set out in Sambrook & Russell, Molecular Cloning: A Laboratory Manual, 3rd Ed., CSHL Press, Cold Spring Harbor, N.Y., 2001.

### Strains

Hyphomicrobium EG: This strain is deposited at the CBS Institute under the deposit number NCCB 84101.

Thiobacillus thioparus TK-m: this strain is deposited at the DSMZ under the deposit number DSM 5368.

Rhodococcus rhodochrous IGTS8: this strain is deposited at the ATCC under the deposit number ATCC 53968.

Escherichia coli strain RV308 is deposited at the ATCC under the deposit number ATCC31608.

B. subtilis strain BS154: this strain is deposited at the CBS Institute under the deposit number CBS 363.94.

### LC-MS/MS (Liquid Chromatography-Mass Spectrometry) Analysis

Protein samples containing ˜0.2 mg/ml protein were pre-treated with and without TCA precipitation. For TCA precipitation, the sample was 1:1 mixed with 20% TCA, which was kept at 4° C. for 1 hour, and subsequently centrifuged for 30 min at 20000 rcf and 4° C. After centrifugation, the supernatant was removed and the pellets were washed once with acetone (minus 20° C.) and centrifuged for 10 min at 20000 rcf and 4° C. The supernatant was removed after drying for 10 min and the pellets were resolved in 50 mM NaOH, followed by adding 100 mM NH₄HCO₃ (pH 8). For the sample without TCA treatment, sample was mixed 5:1 with 200 mM NH₄HCO₃ (pH 8).

Before digestion with trypsin (overnight at 37° C.) samples were reduced and alkylated with DTT and IAA. After acidifying using formic acid the samples were analyzed.

The hydrolyzates were analyzed on an Accela UHPLC (Thermo Electron, Breda, The Netherlands) coupled to a LTQ-velos Mass Spectrometer (Thermo Electron, Bremen, Germany). The chromatographic separation was achieved with a 2.1×100 mm 1.8 micrometer particle size, 80 Å pore size, C-18 Eclipse XDB Zorbax column (Agilent Santa Clara, Calif., USA), using a gradient elution with (A) LC-MS grade water containing 0.1% formic acid B) LC-MS grade acetonitrile containing 0.1% formic acid solution (Biosolve BV, the Netherlands) as mobile phases. The 100 min gradient started from 5% B linear increasing to 30% B in 90 min, increasing to 40% B in an additional 20 min., washing with 80% B over 5 min and re-equilibration with 5% B for 12 min. The flow rate was kept at 0.4 ml/min, using an injection volume of 25 μl and the column temperature was set to 50° C.

The mass spectrometry data acquisition was accomplished with a Top 10 data-dependent acquisition using “Chromatography” and “Dynamic exclusion” options and charge states 2 and 3 included only, scanned for m/z range 400-2000. MS/MS experiments were performed with an isolation width set at 3.0, and the normalized collision energy was set to 35.

Database searches were performed using the Sorcerer 2 (Sorcerer™-SEQUEST®) search engine and the Trans Proteome Pipeline (TPP), using trypsin as preferred enzyme. Only proteins identified with a confidence >90% were considered.

The LC-MS/MS data was searched against Hypomicrobium public and DSM proprietary databases (the latter as described in Example 5). No differences were observed between the results of the samples with and without TCA precipitation

### Headspace Analyses

Headspace analyses were carried out using a gas chromatograph with mass detection (type QP 2010) and an autosampler (type AOC 5000 plus, all from Shimadzu Europe GmbH, 's Hertogenbosch, The Netherlands). The GC-column used (30 m×0.25 mm, df=1.00 micrometer) was article number 12653 (Restek via Interscience B.V., Breda, The Netherlands). The injector and detector temperatures were set to 250° C. and 325° C., respectively. The carrier gas (helium) flow rate through the column was 2.0 mL/min. The column temperature was held at 35° C. for 3 min isothermally and then raised by 40° C./min to 200 degrees C. and held for 0.4 min. The headspace vial was heated at 37° C. for 0.5-10-20-30 and 120 min before 0.5 mL of the headspace was injected.

### Example 1

### Identification of the Methyl Mercaptan Oxidase from Hyphomicrobium EG, Thiobacillus thioparus and Rhodocccus thodochrous

Hyphomicrobium EG, Thiobacillus thioparus TK-M and Rhodocccus rhodochrous cells are cultured as set out in Suylen et al., J. General Microbiology (1987), 133, 2989-2997, Gould and Kanagawa, J. General Microbiology (1992), 138, 217-221 and Kim et al. Biotechnol. Bioprocess Eng. (2000), 5, 465-468 under conditions that induce the production of methyl mercaptan oxidase (MM-oxidase) respectively.

MM-oxidase enzyme is then purified from the three strains as set out in Suylen et al., Gould and Kanagawa and Kim et al.

N-terminal amino acid analysis is then used to determine about 5 to 20 amino acids at the N-terminus of the purified MM-oxidases.

This information may be combined with whole genome sequence data on Hyphomicrobium sp MC1 (France Genoscope—Centre National de Seéquençage 2 rue Gaston Crémieux CP5706 91057 Evry cedex http://www.genoscope.cns.fr/spip/, http://www.ncbi.nlm.nih.gov/nuccore/338736863; Submitted (24 Jun. 2011) National Center for Biotechnology, Information, NIH, Bethesda, Md. 20894, USA) and on Rhodococcus (Genome draft of a cholesterol oxidase producing actinobacterium Rhodococcus rhodochrous strain BKS6-46: Unpublished—Kumar, S., Bala, M., Kaur, I., Mayilraj, S., Raghava, G. P. S. Submitted (14 Nov. 2011) Bioinformatics Centre (BIC), Microbial Type Culture Collection and Gene Bank (MTCC), Institute of Microbial Technology (IMTECH), Sector 39-A, Chandigarh, Chandigarh 160036, India—Kumar, S., Bala, M., Kaur, I., Mayilraj, S., Raghava, G. P. S. http://www.ncbi.nlm.nih.gov/Traces/ms/?val=AGVW01).

The exact amino acid and nucleic acid sequences of the Hyphomicrobium and Rhodococcus MM-oxidases may be determined by combining the partial amino acid data obtained from the purified MM-oxidases with the whole genome data.

### Example 2

### Expression of MM-oxidase in Bacillus subtilis

The E. coli B. subtilis shuttle vector pBHA12 is described in (WO2008/000632) and is used for the expression of the MM-oxidase present in the genomes of strains of Hyphomicrobium, Rhodococcus rhodochrous or Thiobacillus thioparus. The MM-oxidase gene is synthesised and at the coding sequence may be modified to match the coding usage of the B. subtilis expression host. The MM-oxidase gene fragment is cloned into pBHA12 which results in the MM-oxidase expression vectors pBHA12-MM-H, pBHA12-MM-RR and pBHA12-MM-TT.

These vectors are transformed into B. subtilis strain BS154 (CBS 363.94) (ΔaprE, ΔnprE, amyE, spo⁻) as described in Quax and Broekhuizen 1994 Appl Microbiol Biotechnol. 41: 425-431. The B. subtilis strains BS154 containing the MM-oxidase expression vectors are named BSU154MMO-H, BSU154MMO-RR and BSU154MMO-TT.

### Example 3

### Expression of MM-oxidase with B. subtilis in shake flasks

The B. subtilis strains BSU154MMO-H, BSU154MMO-RR and BSU154MMO-TT are grown in shake flasks. These shake flasks contain 20 ml 2×TY medium composed of 1.6% (w/v) Bacto tryptone, 1% (w/v) Yeast extract and 0.5% (w/v) NaCl. The cultures are shaken vigorously at 37° C. and 250 rpm for 16 hours and 0.2 ml culture medium is used to inoculate 20 ml SMM medium. SMM pre-medium contains 1.25% (w/w) yeast extract, 0.05% (w/w) CaCl2, 0.075% (w/w) MgCl2.6H2O, 15 μg/l MnSO4.4H2O, 10 μg/l CoCl2.6H2O, 0.05% (w/w) citric acid, 0.025% (w/w) antifoam 86/013 (Basildon Chemicals, Abingdon, UK). To complete SMM medium, 20 ml of 5% (w/v) maltose and 20 ml of a 200 mM Na-phosphate buffer stock solution (pH 6.8), both prepared and sterilized separately, are added to 60 ml SMM pre-medium. These cultures are incubated for 48 hours at 37° C. and 250 rpm. The cells are harvested by centrifugation 5 minutes at 13000 rpm. The cell pallet is taken up in a buffer containing 10 mM Tris-HCl (pH 7.5), 10 mM EDTA, 20% sucrose, 50 mM NaCl, 1 mg/ml lysozyme and protease inhibitors (Complete EDTA-free protease inhibitor cocktail, Roche). The re-suspended pellets are incubated for 30 minutes at 37° C., and the obtained protoplasts are disrupted by sonication. After sonication the cell debris is spun down by centrifugation 10 minutes at 13000 rpm and the MM-oxidase is measured in the clear lysate.

### Example 4

### Purification of MMO from B. subtilis

The purification of MM-oxidases from the culture lysates obtained in Example 3 is performed using the protocols as set out in Suylen et al., Gould and Kanagawa and Kim et al.

The protein concentration of the purified enzymes may be determined by BCA™ protein assay kit (Pierce) according to the manufacturer's instruction with the following condition: the ratio of the sample to WR reagent was 1:12, and the absorbance of the mixture was determined at wavelength of 540 nm.

MM-oxidase activity is determined in five different ways, namely by following either the utilization of (i) MM or (ii) O₂, or the formation of (iii) H₂O₂, (iv) formaldehyde or (v) sulphide. Utilization and formation of these substances is monitored as set out in Suylen et al., J. General Microbiology (1987), 133, 2989-2997.

### Example 5

### Determining the Genome Sequence of Hyphomicrobium EG

Hyphomicrobium EG was obtained from CBS (Centraalbureau Schimmelcultures, Baarn, The Netherlands; strain reference number NCCB 84101) and its DNA was isolated for determination of the genomic DNA sequence.

A 5 mL pH 6.0 medium incorporating Na₂HPO₄.7H₂O 7.9 g/L, KH₂PO₄ 1.5 g/L, MgSO₄.7H₂O 1 g/L, (NH₄)₂SO₄ 0.8 g/L, DMSO 0.78 g/L and a spore elements solution, was inoculated with single colony isolate and aerobically cultivated for 4 days at 30° C. After 4 days of growth, the cells were pelleted and resuspended in 50 mL fresh DMSO medium and again cultivated for 4 days at 30° C. The resulting culture was used for genomic DNA purification using the Puregene Yeast/BAct. kitB from Qiagen (Venlo, The Netherlands) according to the manufacturers' protocol for Gram-negative bacteria. The purified DNA was diluted to a final concentration of 100 ng/microliter and sent to BaseClear (Leiden, The Netherlands) for sequence analysis.

The DNA was fragmented (shearing) and DNA adapters were ligated to both ends of the DNA fragments. Two sets of Illumina GAIIx sequence reads were obtained. One set consisted of paired-end reads, spanning a distance of around 275 (+−125) nucleotides. The second set consisted of mate pair reads, spanning a distance of around 4500 nucleotides (+−2100). On all Illumina GAIIx sequence reads a quality filtering was applied based on Phred quality scores. In addition, low quality and ambiguous nucleotides were trimmed off from the remaining reads. The reads were used for ‘De novo’ assembly in the CLC Genomics Workbench (CLC Genomics Workbench version 5.5.1, Aarhus, Denmark). In this way, a set of pre-assembled contigs (contiguous sequences) were obtained. The contigs were arranged further (scaffolding) using SSPACE Premium scaffolder version 2.3 described by Boetzer et al. (Bioinformatics 27:578-579, 2011). The gapped regions within the scaffolds were partially closed in an automated manner using GapFiller version 1.10 (Boetzer and Pirovano (Genome Biology 13:R56, 2012). Annotations were generated using the Progenus GAPS pipeline

### Example 6

### Identification of the Gene Encoding Methyl Mercaptane Oxidase

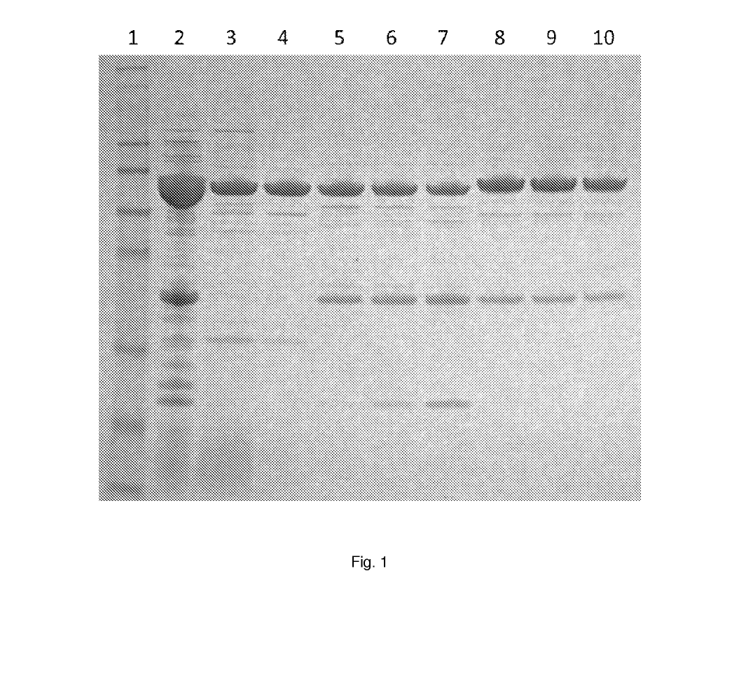

The growth of Hyphomicrobium EG for preparing enzyme extracts was carried out as described by Suylen et al., J. General Microbiology (1987), 133, 2989-2997. After disruption of the wet cell pellet, the crude cell extract was centrifuged and the supernatant was subjected to DEAE chromatography followed by chromatography over hydroxylapatite yielding two protein peaks. Of the two peaks, different chromatographic fractions were collected and subjected to SDS-PAGE (see FIG. 1). Staining was performed using Simply Blue Safe Stain (Collodial Coomassie G250). Activity of the MM-oxidase was tested using the coupled formaldehyde dehydrogenase assay also described in Suylen et al. (supra). The peak fractions depicted in lanes 5, 6 and 7 had a protein content (Bradford) similar to the peak fractions depicted in lanes 8, 9 and 10, but their MM-oxidase activity was twice as high.

The protein peak fractions eluting from the hydroxylapatite column were pooled and analysed by LC-MS/MS using the newly obtained DNA sequence. Details of the LC-MS/MS procedure used are described in the Materials & Methods section. The LC-MS/MS data obtained was searched against two databases:
‘MS01215_{—}Hypomicrobium_sp_MC1_trypsin.fasta’ containing ˜4900 protein sequences of Hipomicrobium publicly known (source: uniprot)
‘MS01215_Iup1_BSA.fasta’ containing ˜3600 protein sequences of the Hyphomicrobium EG strain, the proprietary database generated by DSM—see Example 5.

Database searches were performed using the Sorcerer 2 (Sorcerer™-SEQUEST®) search engine and the Trans Proteome Pipeline (TPP). Only proteins identified with a confidence >90% were considered. No differences were observed between the results of the samples with and without TCA precipitation.

Around 90 different proteins from Hyphomicrobium were identified in the two hydroxylapatite peak fraction samples provided. Using public data base MS01215_{—}Hypomicrobium_sp_MC1_trypsin.fasta. [Hyphomicrobium denitrificans ATCC 51888]′, protein HDEA00762, i.e. Selenium-binding protein 1-A, was identified as the most abundant protein in both fractions, Based on the LC-MS/MS signals measured this protein has an abundance of >50%. The theoretical molecular weight of this protein is 45 KDa. This corresponds to the protein band observed on gel (˜48 kDa).

Using our newly obtained, proprietary Hyphomicrobium EG database (see Example 5), MS01215_Iup1_BSA.fasta, HDEA00789 ‘selenium-binding protein putative was identified as the most abundant protein in both samples. This protein has 91% homology to protein HDEA00762.

The sequence coverage yielded yet another prominent enzyme, but only in the newly obtained proprietary database from Hyphomicrobium EG. The latter prominent enzyme, referred to as HDEA00816, also has an estimated molecular weight around 45 kDa and was referred to as a ‘hypothetical new protein’.

### Example 7

### Cloning and Overexpression of Various Hyphomicrobium EG Genes in E. coli

Of the three most prominent enzymes described in Example 6, that is the two newly identified ‘selenium binding’ proteins HDEA00762 (referred to as MMO1) and HDEA00789 (referred to as MMO3) and the newly identified ‘hypothetical protein’ HDEA00816 (referred to MMO2) genes were designed with a codon usage optimized for E. coli and synthesized by DNA2.0 (Menlo Park Calif., USA). Gene MMO2 was synthesized without the putative signal peptide MAFSLGVTPSSA. Gene MMO1 was also synthesized without signal sequence and is referred to as MMO4.

For cloning purposes, a DNA sequence containing a NdeI site CGAGCAT (was introduced at the 5′-end and a DNA sequence containing a stop codon and a AscI and HindIII site TAAAGGCGCGCCCGGGAAGCTTCTCG was introduced at the 3′end. The four resulting synthetic constructs were named MMO1E, MMO2E, MMO3E and MMO4E. The synthetic DNA constructs MMO1E, MMO2E, MMO3E and MMO4E were cloned in an arabinose inducible E. coli expression vector, containing the arabinose inducible promoter P_{BAD} and regulator araC (Guzman J. Bac. 177:4121-4130, 1995), a kanamycin resistance gene Km(R) and the origin of replication ori327 from pBR322 (Watson, Gene. 70:399-403, 1988). These vectors were named pBAD-MMO1E, pBAD-MMO2E, pBAD-MMO3E, and pBAD-MMO4E and transformed to Escherichia coli strain RV311. Strain RV311 was constructed by deleting the ampC and araB genes from E. coli strain RV308. This resulted in RV311 (su, ΔlacX74, gal IS II::OP308, lacIq⁻, ΔampC and ΔaraB). Strains of RV311 containing pBAD-MMO1E, pBAD-MMO2E, pBAD-MMO3E, and pBAD-MMO4E were named MMO101, MMO102, MMO103 and MMO104, respectively.

### Example 8

### Preparation of the Enzyme Samples from MMO101, MMO102, MMO103 and MMO104

Strains MMO101, MMO102, MMO103 and MMO104 were pre-cultured in 24 well pre-sterile deep well plates (Axygen, CA, USA) containing 2 ml 2×PY medium existing of 1.6% (w/w) Tryptone peptone, 1% (w/w) yeast extract, 0.5% (w/w) NaCl, and 100 μg/ml neomycin. Plates were covered by a Breathseal (Greiner bio-one, Frickenhausen, Germany) and incubated overnight at 30° C., 550 rpm and 80% humidity in a Microton incubator shaker (Infors AG, Bottmingen, Switzerland). Form these pre-cultures 20 μl was used to inoculate a second 24 well pre-sterile deep well plates (Axygen, CA, USA) containing 1.34 ml magic medium (Invitogen, CA, USA) and 0.66 ml 2% yeast extract.

After incubating the cultures for 4 hours at 30° C., 550 rpm and 80% humidity, the cultures were induced with 75 μl 0.62% L-arabinose. After 24 hours incubation at 20° C., 550 rpm, and 80% humidity the cultures were harvested by centrifugation for 10 minutes at 2750 rpm. The cell pallets were stored overnight at −20° C. The cell pallets from the 2 ml cultures were suspended in 1 ml lysis buffer and incubated for one hour at 37° C. Lysis buffer (100 mL) contains (5 ml 1 M Tris HCL, 1 ml DNase I (100 mg/10 mL), 0.2013 g Lyzosyme, 0.5 ml 5 mM MgSO₄. The lysates were centrifuged at 2750 rpm for 10 minutes and the supernatants were removed and stored. The supernatant samples with equal protein content from the resulting supernatants were then tested for MM-oxidase activity in the coupled formaldehyde dehydrogenase assay, basically as described by Suylen et al. but catalase (1.5 BU/ml, DSM Food Specialities, Delft, The Netherlands) was added to prevent the build-up of H2O2.

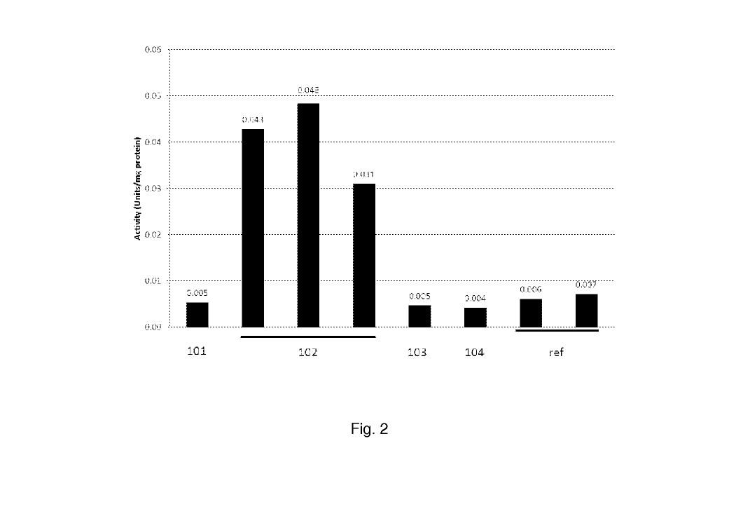

According to the results obtained, only supernatant obtained from the E. coli culture overexpressing gene HDEA00816 (MMO2) showed significant MM-oxidase activity (see FIG. 2). Thus, gene HDEA00816 (MMO2) encodes the desired MM-oxidase.

The nucleotide sequence of the open reading frame named MMO2 is set out in SEQ ID NO: 1 and encodes the HDEA00816 protein, without the putative signal peptide MAFSLGVTPSSA, which is set out in SEQ ID NO: 2. SEQ ID NO: 3 sets out the nucleotide sequence of the E. coli optimized MMO2 gene (named MMO2E) with NdeI site introduced at the 5′-end and a stop codon and AscI and HindIII sites introduced at the 3′end.

### Example 9

### Use of MM-Oxidase in a Chewing Gum

Typical chewing gum compositions prepared in accordance with the present invention are set forth in Table 1. The chewing gums is prepared and the MM-oxidase, for example MMO2 as described in Examples 6 to 8, encapsulated as detailed herein. Suitable flavors and coolants are generally known in the art. Values are % by weight of the chewing gum composition or encapsulated enzyme.

TABLE 1

| Ingredient | 1 | 2 | 3 | 4 | 5 |
|---|---|---|---|---|---|
| Gum base | 34.27 | 26.22 | 41.00 | 42.00 | 32.72 |
| Lecithin | 0.17 | 0.17 | 0.17 | 0.17 | 0.05 |
| Sorbitol | 50.06 | 49.86 | 30.00 | 35.00 | |
| Xylitol | | | | | 60.00 |
| Maltitol | | | 4.00 | 15.00 | |
| Calcium | 10.00 | 17.5 | 20.33 | 1.08 | |
| carbonate | | | | | |
| Glycerin | | 4.00 | 1.00 | | |
| Sorbitol syrup | 2.50 | | | 4.50 | |
| Corn syrup | | | | | 5.0 |
| High intensity | 0.60 | 0.60 | 0.60 | 0.30 | 0.30 |
| sweetener | | | | | |
| Encapsulated | | | | 0.30 | 0.30 |
| high intensity | | | | | |
| sweetener | | | | | |
| Coolant | 0.30 | | 0.30 | | |
| Encapsulated | 0.50 | 0.05 | 1.00 | 0.075 | 0.03 |
| MM-oxidase | | | | | |
| (30% wt | | | | | |
| enzmye based | | | | | |
| on wt. of | | | | | |
| encapsulated | | | | | |
| enzyme) | | | | | |
| Flavour | 1.60 | 1.60 | 1.60 | 1.60 | 1.60 |
| Total | 100.00% | 100.00% | 100.00% | 100.00% | 100.00% |

### Example 10

### Determination of Efficacy of an MM-Oxidase Containing Chewing Gum in Breath Odour Control

In order to determine the efficacy of an MM-oxidase containing chewing gum in controlling breath odour. Mouth breath is scored organoleptically and the MM content determined prior to and following 30 minutes chewing a chewing gum prepared according to Example 9. Organoleptic and VSC measurements are carried out as set out in Tangerman and Winkel (J Clin. Perodontol. 34, 748-755, 2007).

### Example 11

### MM-Oxidase Activity in Toothpaste

To test the compatibility of Hyphomicrobium MMO2 with toothpaste, the following headspace measuring experiment was carried out.

Part of the two protein peaks eluted from the hydroxyapatite column (see Example 6) was freeze dried and equal protein quantities of both peak fractions were mixed with 5 grams of ‘Zendium Classic’ toothpaste (Unilever, UK). A total of 0.25 ml of each mixture was weighed into a 20 ml headspace vial together with 1 ml of water and the vial was firmly closed with a septum and cap. After mixing, to each vial, 0.5 g of calibration standard was added with a syringe through the septum. The calibration standard was prepared by bubbling methyl mercaptan (Sigma Aldrich) through an accurately weighted amount of water for 15 minutes. After bubbling, the concentration of methyl mercaptan was determined by the increase in weight. The resulting stock solution was diluted with different amounts of water to compose different calibration standards. One of these calibration standards was used to add to the Zendium-enzyme mixtures to quantify the degradation of methyl mercaptan over time. The headspace of the calibration standards were analyzed after heating for 5 min at 37° C.

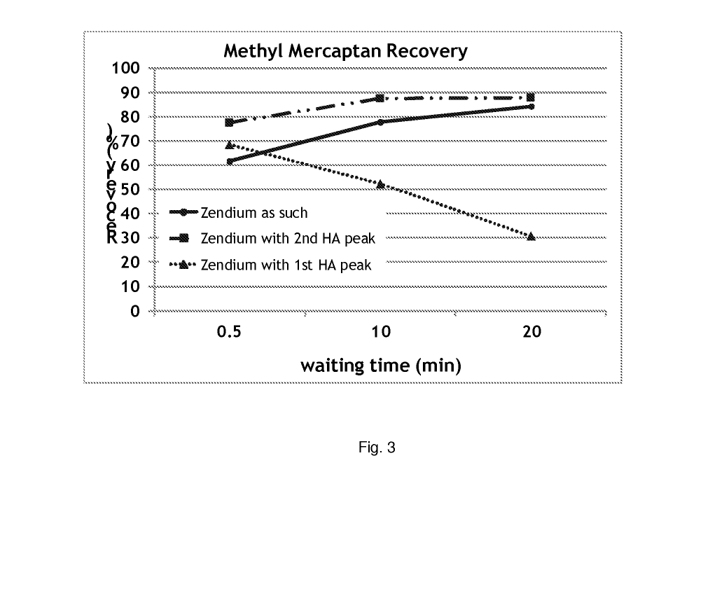

Table 2 lists the results obtained with various chromatographic samples obtained from Hyphomicrobium EG. In FIG. 3 the results are graphically depicted. It is apparent that:
the MM-oxidase present in Hyphomicrobium EG remains active upon mixing with toothpaste;
the MM-oxidase activity is concentrated in the first hydroxyapatite protein fraction (incorporating the major part of protein HDEA00816: MMO2); and
the activity of the MM-oxidase is compatible with a toothpaste formulation as provided by Zendium.

TABLE 2
Compatibility of MM-oxidase with Zendium classic toothpaste

| | Waiting | Methyl Mercaptan | | | Standard |
|---|---|---|---|---|---|
| | time | added | analyzed | recovery | normalized |
| Sample | (min) | (μg) | (μg) | (%) | (%) |
| Cal standard | 0.5 | 8.59 | 7.64 | 89 | 100 |
| Zendium + 2^{nd} HA peak | 0.5 | 8.61 | 5.94 | 69 | 78 |
| Zendium + 1^{st} HA peak | 0.5 | 8.62 | 5.26 | 61 | 68 |
| Zendium as such | 0.5 | 8.46 | 4.64 | 55 | 62 |
| Cal standard | 10 | 8.25 | 8.28 | 100 | |
| Zendium + 2^{nd} HA peak | 10 | 9.13 | 7.98 | 87 | |
| Zendium + 1^{st} HA peak | 10 | 8.39 | 4.38 | 52 | |
| Zendium as such | 10 | 8.69 | 6.76 | 78 | |
| Cal standard | 20 | 8.52 | 8.54 | 100 | |
| Zendium + 2^{nd} HA peak | 20 | 8.53 | 7.49 | 88 | |
| Zendium + 1^{st} HA peak | 20 | 8.71 | 2.67 | 31 | |
| Zendium as such | 20 | 8.64 | 7.29 | 84 | |

1. An oral composition comprising a volatile sulphur compound breath-odour controlling amount of methyl mercaptan oxidase.

2. An oral composition according to claim 1, wherein the volatile sulphur compound is methyl mercaptan or hydrogen sulphide.

3. An oral composition according to claim 1 comprising a zinc salt, optionally comprising an organic zinc salt.

4. An oral composition according to claim 1 which is a liquid or a solid composition.

5. An oral composition according to claim 1 which is a chewing gum, chewing candy, pet chew or biscuit, dentifrice, mint, low boiled candy, hard boiled candy, lozenge, syrup, pressed mint, throat drop or chocolate.

6. An oral composition according to claim 5, wherein the dentifrice is a toothpaste or a toothpowder.

7. An oral composition according to claim 4, which comprises:
a water insoluble base;
a water soluble portion;
a volatile sulphur compound breath-odour controlling amount of methyl mercaptan oxidase; and, optionally
a sweetener and/or a flavour.

8. An oral composition according to claim 3, wherein the methyl mercaptan oxidase is encapsulated.

9. An oral composition according to claim 3 which comprises up to about 5% (w/w) of methyl mercaptan oxidase.

10. An oral composition according to claim 1, wherein the methyl mercaptan oxidase is:
(a) a polypeptide comprising an amino acid sequence as set out in SEQ ID NO: 2;
(b) a polypeptide comprising an amino acid sequence having at least 50% sequence identity with the amino acid sequence of SEQ ID NO: 2;
(c) a polypeptide encoded by a polynucleotide comprising the polynucleotide sequence as set out in SEQ ID NO: 1 or SEQ ID NO: 3; or
(d) a fragment of a polypeptide as defined in (a), (b) or (c).

11. An oral composition according to claim 1, wherein the methyl mercaptan oxidase is a polypeptide comprising an amino acid sequence having at least 60% sequence identity, optionally at least about 70%, optionally at least about 80%, optionally at least about 90%, optionally at least about 93%, optionally at least about 95%, optionally at least about 96%, optionally at least about 97%, optionally at least about 98% optionally at least about 99% sequence identity to SEQ ID NO 2.

12. An oral composition according to claim 1, wherein the methyl mercaptan oxidase is derived from a Hyphomicrobium species, a Thiobacillus species or a Rhodococcus species.

13. An oral composition according to claim 12, wherein the Hyphomicrobium species is Hyphomicrobium EG, the Thiobacillus species is Thiobacillus thioparus or the Rhodococcus species is Rhodococcus rhodochrous.

14. An oral composition according to claim 1, which does not comprise an anti-bacterial component.

15. A method for controlling breath-odour in a host which method comprises:
providing a composition according to claim 1; and
applying the composition to an oral cavity of the host.

16. A method according to claim 15, wherein the composition is a chewing gum and the method comprises chewing the chewing gum to cause the methyl mercaptan oxidase to be released from the chewing gum into the oral cavity.

17. An oral composition according to claim 1 for controlling breath-odour.

18. An oral composition according to claim 1 capable of being used in manufacture of a medicament for use in controlling breath-odour.

19. A polypeptide having methyl mercaptan oxidase activity, wherein said polypeptide is:
(a) a polypeptide comprising an amino acid sequence as set out in SEQ ID NO: 2;
(b) a polypeptide comprising an amino acid sequence having at least 50% sequence identity with the amino acid sequence of SEQ ID NO: 2;
(c) a polypeptide encoded by a polynucleotide comprising the polynucleotide sequence as set out in SEQ ID NO: 1 or SEQ ID NO: 3; or
(d) a fragment of a polypeptide as defined in (a), (b) or (c).

20. A polypeptide according to claim 19, comprising a polypeptide having an amino acid sequence having at least 60% sequence identity, optionally at least about 70%, optionally at least about 80%, optionally at least about 90%, optionally at least about 93%, optionally at least about 95%, optionally at least about 96%, optionally at least about 97%, optionally at least about 98% optionally at least about 99% sequence identity to SEQ ID NO 2.

21. A polynucleotide sequence encoding a polypeptide according to claim 19.

22. A polynucleotide according to claim 21, wherein the polynucleotide is:
(a) a polynucleotide comprising the sequence set out in SEQ ID NO: 1 or SEQ ID NO: 3 or comprising a sequence having at least 50% sequence identity with the polynucleotide sequence of SEQ ID NO: 1 or SEQ ID NO: 3;
(b) a polynucleotide which is degenerate as a result of the degeneracy of the genetic code to a polynucleotide sequence as defined in (a); or
(c) a polynucleotide which is the reverse complement of a nucleotide sequence as defined in (a) or (b).

23. A polynucleotide sequence according to claim 22, having a sequence identity of at least 60%, optionally at least 70%, optionally at least 80%, optionally at least 90%, optionally at least 93%, optionally at least about 95%, optionally at least about 96%, optionally at least about 97%, optionally at least about 98%, optionally at least 99% to SEQ ID NO: 1 or 3.

24. A nucleic acid construct comprising the polynucleotide sequence of claim 21.

25. A nucleic acid construct according to claim 24 which is an expression vector, wherein the polynucleotide sequence is operably linked to at least one control sequence for the expression of the polynucleotide sequence in a host cell.

26. A host cell comprising the polynucleotide according to claim 21.

27. A host cell according to claim 26 which is a prokaryotic host cell, optionally a prokaryotic cell being a bacterial cell, or an eukaryotic host cell, optionally an eukaryotic host cell selected from a mammalian, insect, plant, fungal, or algal cell.

28. A method of producing a polypeptide of claim 19, comprising:
(a) cultivating the host cell under conditions conducive to the production of the polypeptide by the host cell; and, optionally,
(b) recovering the polypeptide.

29. A composition, such as an oral composition, comprising a polypeptide according to claim 19 and at least one additional ingredient.
